(19) United States
(12) Patent Application Publication
(10) Pub. No.: US 2010/0166721 A1
(43) Pub. Date: Jul. 1, 2010

(54) PROBOTIC COMPOSITIONS AND USES THEREOF

(76) Inventor: Fabiola Masri, Quebec (CA)

Correspondence Address: OGILVY RENAULT LLP, 1, Place Ville Marie, SUITE 2500, MONTREAL, QC, H3B 1R1, CA

(21) Appl. No.: 12/303,847

(22) PCT Filed: Jun. 11, 2007
(86) PCT No.: PCT/CA07/01030

§ 371 Date: Dec. 8, 2008

Related U.S. Application Data
(60) Provisional application No. 60/804,329, filed on Jun. 9, 2006.

(51) Int. Cl. A61K 35/74; A61P 1/14; A23L 1/30

(52) U.S. Cl. 424/93.44; 426/61

(57) ABSTRACT

The present application relates to a probiotic composition comprising a probiotic bacteria. The composition is particularly useful as a food supplement for normalizing the gastro-intestinal flora. The probiotic bacteria present in the composition contain species of Propionibacterium, Lactobacillus, Bifidobacterium and Streptococcus. Also disclosed herein are methods of producing such composition and uses of the composition.

### RELATED APPLICATIONS

This application claims priority from U.S. patent application 60/804,329 filed Jun. 9, 2006, the specification of which is hereby incorporated by reference.

### FIELD OF THE INVENTION

The present invention relates to probiotic compositions comprising living bacteria selected from the group of propionibacteria, lactic acid bacteria (such as lactobacilli), bifidobacteria and streptococcus. Specific formulations are provided for specific uses, including the use as a food supplement. Because of their probiotic nature, the compositions are particularly useful in human and animal health. These compositions are further particularly useful in the restoration of a gastrointestinal flora as well as in the prevention and treatment of gastrointestinal disorders.

### BACKGROUND OF THE INVENTION

Probiotics are living bacteria that, when consumed in sufficient quantities, are beneficial to the health of the host. These useful bacteria can fight and neutralize pathogenic bacteria, particularly in the gastro-intestinal (GI) tract. Probiotic supplements are mainly dietary supplements containing or derived from potentially beneficial bacteria or yeast. Lactic acid bacteria (LAB) have been used in the food industry for many years as probiotics. LAB are able to convert lactose into lactic acid. This not only provides the characteristic sour taste of fermented dairy foods such as yogurt, but acts as a preservative, by lowering the pH and limiting the growth of spoilage or pathogenic organisms.

Probiotic bacterial cultures are intended to assist the body's naturally occurring flora within the digestive tract to reestablish themselves. They are sometimes recommended by doctors, and, more frequently, by nutritionists, after a course of antibiotics, or as part of the treatment for candidiasis. Many probiotics are present in natural sources such as lactobacilli in yogurt and sauerkraut.

The rationale for probiotics is that the gut contains a miniature ecology of microbes, collectively known as the gut flora. The number and types of bacteria present in a healthy gut flora limits or prevents the growth of pathogenic microorganisms. The bacterial balance of the gut flora can be shifted by wide range of circumstances including the use of antibiotics or other drugs, excess alcohol intake, stress, diseases and disorders, exposure to toxic substances, or even the use of antibacterial soap. In cases like these, the number or type of healthy gut flora bacteria may be modulated, an event which may allow harmful competitors to thrive (such as pathogenic bacteria), to the detriment of the human or animal health.

There is no published evidence that probiotic supplements are able to replace the body's natural flora that has been reduced or eliminated. There is evidence, however, that probiotics do form beneficial temporary colonies which may assist the body in the same functions as the natural flora, while allowing the natural flora time to recover from depletion. The probiotic species are then progressively replaced by a naturally developed gut flora. If the conditions which originally caused damage to the natural gut flora persist, the benefits obtained from probiotic supplements will be short lived.

Scientists have found a range of potentially beneficial medicinal uses for probiotics, such as the management of lactose intolerance. Because lactic acid bacteria convert lactose into lactic acid, their ingestion may help lactose intolerant individuals tolerate more lactose than what they would otherwise. Probiotics could also play a role in the prevention of colon cancer since, in laboratory investigations, lactic acid bacteria have demonstrated anti-mutagenic effects thought to be due to their ability to bind with (and therefore detoxify) heterocyclic amines; carcinogenic substances formed in cooked meat. Animal studies have demonstrated that lactic acid bacteria can protect against colon cancer in rodents. Most human trials have found that lactic acid bacteria may exert anti-carcinogenic effects by decreasing the activity of an enzyme called β-glucuronidase, which can regenerate carcinogens in the digestive system. Lower rates of colon cancer among higher consumers of fermented dairy products have been observed in some population studies; the results of which are encouraging.

Probiotics could also exert an effect on the circulatory system by lowering cholesterol levels or blood pressure. Animal studies have demonstrated the efficacy of a range of lactic acid bacteria to lower serum cholesterol levels, presumably by breaking down bile in the gut, thus inhibiting its reabsorption. Some human trials have shown that dairy foods fermented with lactic acid bacteria can produce modest reductions in total and LDL cholesterol levels in those with normal levels to begin with, however trials in hyperlipidemic subjects are needed. Further, several small clinical trials have shown that consumption of milk fermented with various species of lactic acid bacteria can result in modest reductions in blood pressure. It is thought that this is due to the ACE inhibitor-like peptides produced during fermentation.

In addition, probiotics have been shown to have a role on the immune system. Lactic acid bacteria are thought to have several beneficial effects on immune function. They may protect against pathogens by means of competitive inhibition (i.e. by competing for growth) and there is evidence to suggest that they may improve immune function by increasing the number of IgA-producing plasma cells, increasing or improving phagocytosis as well as increasing the proportion of T lymphocytes and Natural Killer cells. Clinical trials have demonstrated that probiotics may decrease the incidence of respiratory tract infections and dental cavities in children as well as help in the treatment of Helicobacter pylori infections (which cause peptic ulcers) in adults when used in combination with standard medical treatments. Lactic acid bacteria foods and supplements have been shown to be effective in the treatment and prevention of acute diarrhea, decreasing the severity and duration of rotavirus infections in children as well as antibiotic associated and travelers diarrhea in adults. Lactic acid bacteria-containing foods and supplements have also been found to modulate inflammatory and hypersensitivity responses, an observation thought to be at least in part due to the regulation of cytokine function. Clinical studies suggest that they can prevent recurrences of Inflammatory Bowel Disease (IBD) in adults, as well as improve milk allergies and decrease the risk of atopic eczema in children.

While probiotics can provide a wide variety of benefits, it should be noted that their efficiency level can vary greatly from one strain to another. Selecting and dosing bacterial strains while preparing a probiotic supplement is of crucial importance that will ultimately lead to the efficiency or the non-efficiency of the final product. Probiotics must therefore be carefully chosen based on, amongst other factors, their viability, stability, gastric acid resistance, biliary salts resistance, and their compatibility with one another.

### SUMMARY OF THE INVENTION

The present invention relates to probiotics compositions comprising a high concentration of probiotic bacteria.

According to one aspect of the present invention, there is provided a probiotic composition comprising a mixture of living bacteria comprising a Propionibacterium, a Lactobacterium, a Bifidobacterium, and a Streptococcus.

According to a further aspect of the present invention, there is provided a probiotic composition comprising a mixture of living bacteria comprising a Lactobacterium selected from the group comprising Lactobacillus acidophilus, Lactobacillus casei, Lactobacillus plantarum, Lactobacillus paracasei, Lactobacillus rhamnosus, Lactobacillus bulgaricus, Lactobacillus reuteri, Lactobacillus salivarius, and Lactobacillus crispatus; a Bifidobacterium selected from the group comprising Bifidobacterium longum, Bifidobacterium lactis, Bifidobacterium bifidum, Bifidobacterium infantis, and Bifidobacterium breve; a Streptococcus being Streptococcus thermophilus; and a Propionibacterium being Propionibacterium freudenreichii.

According to another aspect of the invention, there is provided a probiotic composition comprising 10 to 30% of Lactobacillus acidophilus, 5 to 30% of Lactobacillus paracasei, 5 to 30% of Bifidobacterium lactis, 5 to 30% of Bifidobacterium longum, 5 to 20% of Bifidobacterium bifidum, 5 to 20% of Bifidobacterium breve, 5 to 30% of Bifidobacterium infantis, 5 to 30% of Propionibacterium freudenreichii, and 2 to 20% of Streptococcus thermophilus.

According to another aspect of the invention, there is provided a probiotic composition comprising 10 to 30% of Lactobacillus rhamnosus, 10 to 30% of Lactobacillus acidophilus, 10 to 30% of Lactobacillus paracasei, 10 to 30% of Lactobacillus plantarum, 5 to 20% of Bifidobacterium bifidum, 5 to 20% of Bifidobacterium breve, 5 to 15% of Streptococcus thermophilus, and 5 to 20% of Propionibacterium freudenreichii.

According to another aspect of the invention, there is provided a probiotic composition comprising 10 to 30% of Lactobacillus rhamnosus, 10 to 30% of Lactobacillus plantarum, 10 to 30% of Lactobacillus acidophilus, 5 to 20% of Lactobacillus paracasei, 5 to 20% of Propionibacterium freudenreichii, 5 to 20% of Bifidobacterium lactis, 5 to 10% of Bifidobacterium bifidum, 5 to 10% of Bifidobacterium breve, 5 to 15% of Bifidobacterium infantis, 3 to 10% of Bifidobacterium longum, 2 to 10% of Lactobacillus bulgaricus, and 2 to 10% of Streptococcus thermophilus.

According to yet another aspect of the invention, there is provided a probiotic composition comprising a Propionibacterium, a Lactobacterium, a Bifidobacterium, a Streptococcus and a prebiotic such as, for example, inuline or oligofructose.

According to an aspect of the present invention, the probiotic composition comprises from between 10⁸ to 10¹² probiotic bacteria. According to another aspect of the present invention, the probiotic bacteria concentration in the probiotic composition remains substantially constant during storage of the probiotic composition.

According to a further aspect of the invention, there is provided a method for normalizing the gastrointestinal flora in a subject in need by administering a probiotic composition to the subject. According to yet a further aspect of the invention, there is provided a method for preventing and treating a gastrointestinal disorder in a subject in need thereof by administering a probiotic composition to the subject. According to still a further aspect of the invention, there is provided a use of a probiotic composition for the manufacture of a preparation for the normalization of the gut flora, or for the manufacture of a preparation for the prevention and treatment of gastrointestinal disorders in a subject.

According to another aspect of the invention, there is provided a probiotic composition comprising a mixture of a Propionibacterium and at least one other probiotic bacteria species. According to a further aspect of the invention, the other probiotic bacteria species is selected from the group comprising Lactobacillus, Bifidobacterium, and Streptococcus.

In the following description of the present product, terms and expressions are used in the following manner;

The term “probiotic” is intended to mean a microorganism (such as a bacteria or a yeast) having a beneficial effect on the general health of an animal or a human, or a beneficial effect on a specific health problem, disorder or disease, alleviating pain, symptoms or discomfort associated with those health problem, disorder or disease.

The expression “composition” is intended to mean a general product comprising at least one probiotic. It will be understood that this expression encompass a variety of specific products, all presenting the characteristic of comprising at least one probiotic.

The expression “dairy-derived composition” is intended to mean that the composition comprises at least one dairy product or a derivative of a dairy product. A derivative of a dairy-product is a product obtained form the transformation of milk, such as, in a non-limitative manner, powder milk, condensed milk, cream, buttermilk, cheese, and yogurt. Preferably, the milk is bovine milk, but it can also originate from, for example, goat, sheep, water buffalo or yak.

The term “prebiotic” as used herein is intended to encompass food ingredients beneficially affecting the host by selectively stimulating growth and/or activity of at least one gastro-intestinal bacteria, including probiotic bacteria. Examples of prebiotics that can be used according to an aspect of the present invention include inulin and oligosaccharides such as fructooligosaccharides, xylooligosaccharides and galactooligosaccharides.

The term “about” as used herein is intended to reflect a variation of 10% of the value it is attached to. For example, a concentration of “about 20%” is reflective of a concentration ranging from 18% to 22%. As another example, a quantity of “about 10⁸” is reflective of a range of 0.9×10⁸ to 1.1×10⁸.

The terms “gut flora”, “gastric flora”, “intestinal flora” and “gastrointestinal flora” as used herein are interchangeable and are intended to represent the normal, naturally occurring bacterial population present in the gastric and intestinal systems of a healthy animal or human. It is meant to reflect both the variety of bacterial species and the total number (or concentration) of bacterial species normally found in a healthy animal or human.

The expression “reconstituting the gastrointestinal flora” as used herein is intended to mean the reappearance of normally occurring microbial species that had vanished or decreased in quantity from the gastrointestinal system, or their increase in number or concentration to levels comparable with those of a healthy animal or human. The expression “stabilizing the gastrointestinal flora” as used herein is intended to mean the limiting of variations in the microbial composition of the gastrointestinal flora, or in the microbial concentrations of the gastrointestinal flora. The expression “normalizing the gastrointestinal flora” as used herein is intended to mean the adjustment of the microbial species variety and concentration levels to those normally encountered in a healthy animal or human.

The expression “gastrointestinal disorders” as used herein is intended to reflect on all gastrointestinal disorders in which an unbalance or deregulation of the normal gut flora is a cause or a symptom. Examples of such gastrointestinal disorders include, in a non-limitative manner, inflammatory bowel disease, ulcerative colitis, Crohn's disease, infectious enteritis, antibiotic-associated diarrhea, diarrhea, colitis, colon polyps, familial polyposis syndrome, Gardner's Syndrome, helicobacter pylori infection, irritable bowel syndrome, and intestinal cancers.

The terms “administration” and the likes as used herein are intended to encompass any active or passive administration of the composition according to the present invention to the gastrointestinal tract, such as, for example, oral and rectal administration. The terms “dose” and “dosage” as used herein are interchangeable and are intended to mean the quantity of the present product to be taken or absorbed at any one time. The term “preparation” as used herein is intended to reflect on all formulation and preparation suitable for efficiently administrating the composition according to the present invention to the gastrointestinal tract.

The expression “food supplement” as used herein is intended to mean a probiotic supplement to be added to, or taken along with, any suitable type of food.

The expression “personal hygiene” as used herein is intended to mean the sanitary practices concerning the maintenance of health and the prevention of illness.

The terms “propionic bacteria”, “propionibacteria” and “Propionibacterium” as used herein are interchangeable and are intended to mean bacteria capable of producing propionic acid.

The terms “lactic acid bacteria”, “lactobacteria” and “lactobacterium” (also referred to as LAB) as used herein are interchangeable and are intended to mean a bacteria capable of producing lactic acid.

### BRIEF DESCRIPTION OF FIGURES

FIG. 1 illustrates the TIM-1 gastro-intestinal model, with the stomach (1), duodenum (2), jejunum (3), ileum (4), gastric secretions (5), and gut secretions (6) mainly composed of bicarbonate, bile and pancreatic juice. The model also contains a peristaltic pump (7) and a dialysis system (8) for the jejunum (3) and ileum (4).

FIG. 2 illustrates the population of lactobacilli during GI transit in the stomach, duodenum, jejunum and ileum. ♦=stomach, x=duodenum, Δ=jejunum, ∘=ileum.

FIG. 3 illustrates the population of bifidobacteria during GI transit in the stomach, duodenum, jejunum and ileum. ♦=stomach, x=duodenum, Δ=jejunum, ∘=ileum.

FIG. 4 illustrates the stability of combined populations of lactobacilli and bifidobacteria in BIACTIVE TOURISTA™ supplement over time. Units are expressed in billions CFU/capsule.

FIG. 5 illustrates the stability of combined populations of lactobacilli and bifidobacteria in BIACTIVE JUNIOR™ supplement over time. Units are expressed in billions CFU/capsule.

FIG. 6 illustrates the stability of combined populations of lactobacilli and bifidobacteria in BIACTIVE 12™supplement over time. Units are expressed in billions CFU/capsule.

FIG. 7 illustrates the fabrication procedure of a functional beverage comprising probiotic bacteria.

FIG. 8 illustrates the survival of probiotic bacteria in the stomach over time. Units are expressed in CFU/g. ♦=S. thermophilus, ▪=Lb. acidophilus, ▴=Lb. casei, x=Lb. plantarum, *=Bifidobacterium.

FIG. 9 illustrates the survival of probiotic bacteria in the duodenum over time. Units are expressed in CFU/g. ♦=S. thermophilus, ▪=Lb. acidophilus, ▴=Lb. casei, x=Lb. plantarum, *=Bifidobacterium.

FIG. 10 illustrates the survival of probiotic bacteria in the jejunum over time. Units are expressed in CFU/g. ♦=S. thermophilus, ▪=Lb. acidophilus, ▴=Lb. casei, x=Lb. plantarum, *=Bifidobacterium.

FIG. 11 illustrates the survival of probiotic bacteria in the ileum over time. Units are expressed in CFU/g. ♦=S. thermophilus, ▪=Lb. acidophilus, ▴=Lb. casei, x=Lb. plantarum, *=Bifidobacterium.

### DETAILED DESCRIPTION OF THE VARIOUS ASPECTS

The compositions described herein contain probiotics. It can be used for reconstituting, stabilizing or normalizing the gastrointestinal flora. It also stimulates the immune system. These compositions also possess interesting organoleptic properties, thereby facilitating intake and patient's compliance.

In one aspect of the invention, a probiotic composition is provided comprising a Propionibacterium and at least one other probiotic bacteria. In another aspect of the invention, a probiotic composition comprising 4 different species of probiotic bacteria; Propionibacterium, Lactobacterium, Bifidobacterium and Streptococcus. Those 4 species present compatibility one with another. In a further aspect of the invention, a probiotic composition comprising 7 different probiotic bacterial strains is provided.

Inoculation values, compatibility between the different species and strains and formulations were adjusted starting from the manufacturer of the bacterial species and strains, and adjusted by various fermentation assays as exemplified hereafter. Resistance of probiotic bacteria to the various stresses encountered during gastro-intestinal transit was one of the factor that was tested and used for adjusting. Another example of factor that was taken into consideration is the duration of the milk fermentation process, as well as the temperature at which the fermentation was performed. For the non-dairy formulations such as the capsules compositions, competition between probiotic bacteria was a lesser aspect since the probiotic bacteria are present in the capsules in powder form. For dairy formulations, rapid acidification of milk during fermentation induced competition and antagonism between some of the probiotic bacteria, which required additional experimentation in order to adjust inoculation values and pH, for example, of the final product before storage, as exemplified hereinafter.

In an aspect, the compositions described herein is presented as a nutritional or dietary supplement (e.g. BIACTIVE™). The compositions may be available in different formulations, with variations in the probiotic species, the concentration and proportion of each individual species alone or with one another, the additional bacteria species, and the non-active ingredients (conferring particular properties such as color, flavor, etc.). The formulations will vary according to the intended uses of the compositions. For example, if the compositions are intended to be administered to newborns, they will be formulated to facilitate intake by newborns. If the compositions are intended to replace temporarily the gut flora, they will be formulated to achieve this purpose.

Examples of common probiotic bacteria include, but are not limited to, lactic acid bacteria such as Lactobacillus acidophilus, Lactobacillus casei, Lactobacillus plantarum, Lactobacillus rhamnosus, Lactobacillus GG, Lactobacillus bulgaricus, and Streptococcus thermophilus. Bacteria commonly found in the normal gut flora, such as bifidobacteria, can also be considered as probiotic bacteria, examples of such being, in a non-limitative manner, Bifidobacterium bifidum, Bifidobacterium breve, Bifidobacterium infantis and Bifidobacterium longum.

In addition to lactic acid bacteria and bifidobacteria, propionic acid bacteria (or propionic bacteria, or propionibacteria) can also be considered as probiotics. Propionibacteria are slow-growing, non spore-forming, Gram-positive, anaerobic bacteria that can be rod-shaped or branched and can occur singularly, in pairs, or in groups. Propionic bacteria converts lactate to propionic acid, acetic acid and carbon dioxide (CO₂). They have been studied for their adhesion capacity to the cell epithelium, and for their resistance in the gastro-intestinal tract. Propionibacteria produce fermentation metabolites such as short-chain fatty acids. They also have the capacity to prevent colic cancer, as well as playing a role in cell regeneration, immunomodulation, vitamin and bacteriocin production, and growth stimulation of various species of beneficial gut flora (bifidogenic effect for example).

So far, studies have shown that propionibacteria are resistant to digestive stress and that they adhere to the intestinal epithelium. They increase lactose digestion by having a β-galactosidase activity, they have anti-mutagenic and pro-apoptotic properties, particularly on colon cancer. Animal studies showing the effects of propionibacteria on immunomodulation, lipid metabolism and β-glucuronidase activity have been performed.

Probiotic-containing compositions may be designed to address several needs. The very nature of the composition (the amount and type of bacteria present in the composition) will strongly influence its ability to address these specific needs.

There is a need for probiotics-containing compositions containing a high concentration of probiotic bacteria.

In an aspect, the probiotic bacterial concentration does not vary over storage time. Consequently, the compositions described herein all possess a minimal probiotic content (e.g. between 10⁸ to 10¹² probiotic bacteria per dosage), thereby conferring probiotic efficiency to the compositions. These minimal probiotic content are substantially preserved during the storage of the composition. Storage of the composition can be performed at room temperature, in a refrigerator or in a freezer. When the composition is stored at room temperature, the composition is preferably stored in an air-tight container.

In a further aspect, when the composition comprises more than one species of probiotic bacteria, the ratio between the species does not substantially vary during the storage of the composition.

Storage of the present product is to be performed at room temperature, in a refrigerated space or as a frozen product.

The composition described herein comprises a mixture of four bacterial genus. These probiotic bacterial genus include, but are not limited to, lactic acid bacteria, and propionic acid bacteria. These probiotic bacterial species may be selected from the group consisting of Lactobacillus (e.g. Lb. acidophilus, Lb. casei, Lb. plantarum, Lb. casei paracasei, Lb. rhamnosus, Lb. bulgaricus, Lb. reuteri, Lb. salivarius, Lb. crispatus), Bifidobacterium (e.g. B. longum, B. lactis, B. bifidum, B. infantis, B. breve), Propionibacterium (e.g. P. freudenreichii), Enterococcus (e.g. E. faecium), Lactococcus (e.g. L. lactis) and Streptococcus (e.g. S. thermophilus). In an aspect, each bacteria species is present in a number comprised between 6 and 500 billions bacteria per dose.

The composition can further comprises yeasts (such as Saccharomyces, S. cerevisiae, S. boulardii), as well as commonly used components found in formulations such as vitamins, minerals, oligosaccharides, fructooligosaccharides, water, powder milk, powder skim milk, mashed fruit, juice, vegetarian capsule, maltodextrin, magnesium stearate, magnesium octadecanoate, silica gel, silicon dioxide, cellulose, microcrystalline cellulose, adjuvants, enrobing agents, anti-agglomerate agents, lubricants, etc.

The compositions described herein can be categorized as being a pharmaceutical product, a natural health product, a food product, a dairy-derived product, a fermented milk product, a soy-based product, a beverage, a food supplement, a dietary supplement, a non-pharmaceutical lactic ferment, a personal hygiene product or a cosmetic. Its use can be preventive or curative. It can be presented in various form, such as an energy bar, a meal substitute, a beverage, a tea-based beverage, a herb-based beverage, a fruit juice-based beverage, a milk, a condensed milk, a milk powder, a flavored milk, a milk shake, a yogurt, a fermented milk in lyophilized powder form, a fermented milk in liquid form, a cheese, a cream, a frozen yogurt, an iced cream, a laxative powder, a vegetal fiber, a syrup, an isotonic liquid supplement containing electrolytes, a juice, an energy drink, a fermented food, a cereal, a cookie or a candy.

The compositions described herein can be administered in the form of a liquid or a solid. When they are administered in form of a solid, it may be lyophilized then processed to be administered in form of a capsule, a tablet or a suppository.

The compositions can be administered in various forms, such as, but not limited to a powder, a bulb, a capsule, a pill, a tablet, a liquid, a gel, a cream or an ointment. It can be administrated orally, rectally, intravaginally or transdermally. The present product is destined to human or animal consumption. In humans, it can be administered to a newborn, a baby in-arms, a baby, an infant, a child, a teenager, an adult or an elder. The recommended posology can vary from 1 to 10 doses per day. Duration of treatment is to be determined by a healthcare professional such as, for example, a doctor, a physician, a naturopath, a nutritionist, and a dietician. The treatment can be for a predetermined length of time or until symptoms have vanished. The present product can be taken together with a meal or ingested with dairy products.

The present product are to be used in order to treat or prevent various types of disorders, diseases and syndromes such as, but not limited to: gastro-intestinal disorders and diseases (such as bacterial gastro-enteritis, viral gastro-enteritis, vomiting, diarrhea, post-antibiotic therapy diarrhea, lactose-intolerance-related diarrhea, traveler's diarrhea, irritable bowel syndrome, constipation, ulcers, gastric ulcers, intestinal ulcers, pouchitis, colitis, ulcerative colitis, gastric distension, flatulence, abdominal pain associated with irritable bowel syndrome), skin disorders and diseases (such as acne), immunologic disorders and diseases, urogenital disorders and diseases (such as vaginal infections) and viral infections (such as rotavirus infections). The present product can also be used as a nutritional supplement, in order to stabilize or normalize the gastric flora, without being directed as treating a particular disorder or alleviating particular symptoms.

The present invention will be more readily understood by referring to the following examples which are given to illustrate the invention rather than to limit its scope.

### EXAMPLES

### Example 1

### Resistance of Bacterial Strains to Various Gastro-Intestinal Stresses

Various bacterial species and strains have been tested for their capacity to survive in the gastro-intestinal (GI) tract. Gastric pH usually range from pH=2 with an empty stomach, to pH=3.5 after a meal. Resistance of bacteria was evaluated toward gastric acid (at concentrations from 0.1 to 0.5% in Man, Rogosa and Sharpe media (MRS)), oxgall (which is a purified, dried form of ox bile used in bacteriological media formulations for gastro-intestinal tract organisms, used at a concentration of 0.1 to 0.5% in MRS), sodium thioglycolate (a biliary salt, used at concentrations of 0.1 to 0.5% in MRS), H₂O₂ (bacterial metabolite capable of inhibiting the growth of some bacteria, used at concentrations of 50 to 300 μg/ml in MRS), lysozyme (used at concentrations of 0.1 to 1 mg/ml in MRS). Maximal concentrations in the tested range to which bacterial strains show resistance are presented in table 1.

TABLE 1
Resistance of bacterial strains to various gastro-intestinal stresses.

| | | Gastric | | Sodium | | |
|---|---|---|---|---|---|---|
| Species | Strain | acid | Oxgall | thioglycolate | H₂O₂ | Lysozyme |
| P. acidipropionici | DH 42 | 0.5% | 0.5% | 0.5% | 100 μg/ml | 1 mg/ml |
| P. freudenreichii | P 63 | 0.5% | 0.5% | 0.5% | 50 μg/ml | 1 mg/ml |
| P. acidipropionici | EQ42 | 0.5% | 0.5% | 0.5% | 50 μg/ml | 1 mg/ml |
| P. freudenreichii | R 0501 | 0.5% | 0.5% | 0.5% | 50 μg/ml | 1 mg/ml |
| Lb. rhamnosus | R 0011 | 0.5% | 0.5% | 0.5% | 50 μg/ml | 1 mg/ml |
| Lb. casei | R 0256 | 0.5% | 0.5% | 0.5% | 50 μg/ml | 1 mg/ml |
| Propionibacterium | R 1042 | 0.5% | 0.5% | 0.5% | 50 μg/ml | 1 mg/ml |
| Lb. bulgaricus | mibl-2 | 0.5% | 0.5% | 0.5% | 50 μg/ml | 1 mg/ml |
| Lb. rhamnosus | R 1039 | 0.5% | 0.5% | 0.5% | 50 μg/ml | 1 mg/ml |
| Lb. casei | R 0215 | 0.5% | 0.5% | 0.5% | 50 μg/ml | 1 mg/ml |
| Lb. plantarum | HN | 0.5% | 0.5% | 0.5% | 100 μg/ml | 1 mg/ml |
| Lb. plantarum | R 1078 | 0.5% | 0.5% | 0.5% | 50 μg/ml | 1 mg/ml |
| Lb. plantarum | R 0202 | 0.5% | 0.5% | 0.5% | 50 μg/ml | 1 mg/ml |
| Lb. plantarum | R 1027 | 0.5% | 0.5% | 0.5% | 50 μg/ml | 1 mg/ml |
| Lb. acidophilus | LA-HN | 0.5% | 0.5% | 0.5% | 50 μg/ml | 1 mg/ml |
| Lb. acidophilus | BG2F04 | 0.5% | 0.5% | 0.5% | 50 μg/ml | 1 mg/ml |
| Lb. casei paracasei | CRL-431 | 0.5% | 0.5% | 0.5% | 50 μg/ml | 1 mg/ml |
| Bifidobacterium isolate | — | 0.5% | 0.4% | 0.5% | <50 μg/ml | <0.1 mg/ml |
| S. thermophilus | R 0083 | ND | 0.3% | 0.5% | ND | ND |
| Lb. casei | R 0215 | ND | 0.5% | 0.5% | ND | ND |
| Lb. acidophilus | 150 | ND | 0.5% | 0.5% | ND | ND |
| Lb. delbrueckii spp lactis | R 0187 | ND | 0.5% | 0.5% | ND | ND |
| Lb. acidophilus | R 052 | ND | 0.5% | 0.5% | ND | ND |
| 50S Lb. delbrueckii spp lactis | R 0187 | ND | 0.2% | 0.5% | ND | ND |
| 50L Lb. delbrueckii spp lactis | R 0187 | ND | 0.5% | 0.5% | ND | ND |

ND = Not detectable

### Example 2

### Growth Rate of Bacterial Strains at Different pH Values

Various bacterial species and strains have been tested for their growth capacity in MRS at pH values ranging from 5 to 3.4, representatives of the acidic conditions of fermented products. Results are presented in Table 2.

TABLE 2
Maximal acidic pH value for acceptable growth of bacterial strains

| Species | Strain | pH value |
|---|---|---|
| P. acidipropionici | DH 42 | 4.0 |
| P. freudenreichii | P 63 | 4.0 |
| P. acidipropionici | EQ42 | 4.0 |
| P. freudenreichii | R 0501 | 3.8 |
| Lb. rhamnosus | R 0011 | 3.8 |
| Lb. casei | R 0256 | 3.8 |
| Propionibacterium | R 1042 | 4.0 |
| Lb. bulgaricus | mibl-2 | 3.8 |
| Lb. rhamnosus | R 1039 | 4.0 |
| Lb. casei | R 0215 | 4.2 |
| Lb. plantarum | HN | 4.0 |
| Lb. plantarum | R 1078 | 4.0 |
| Lb. plantarum | R 0202 | 4.2 |
| Lb. plantarum | R 1027 | 4.2 |
| Lb. acidophilus | LA-HN | 3.8 |
| Lb. acidophilus | BG2F04 | 3.8 |
| Lb. casei paracasei | CRL-431 | 4.0 |
| Bifidobacterium isolate | — | 4.4 |
| S. thermophilus | R 0083 | 4.0 |
| Lb. casei | R 0215 | 3.8 |
| Lb. acidophilus | 150 | 3.8 |
| Lb. delbrueckii spp lactis | R 0187 | 3.8 |
| Lb. acidophilus | R 052 | 3.8 |
| 50S Lb. delbrueckii spp lactis | R 0187 | 4.4 |
| 50L Lb. delbrueckii spp lactis | R 0187 | 4.0 |

### Example 3

### Survival of Bacterial Strains Following an Acid Shock

Various bacterial species and strains have been tested for their survival capacity after contact with HCl at pH=2, which is representative of the acidic conditions in the stomach. Results are presented in log CFU/ml in Table 3, after initial contact (t=0), 1 hour (t=1 h), 2 hours (t=2 h) and 3 hours (t=3 h).

TABLE 3
Survival of bacterial strains following exposition to HCl pH = 2.

| Species | Strain | t = 0 | t = 1 h | t = 2 h | t = 3 h |
|---|---|---|---|---|---|
| P. acidipropionici | DH 42 | 6.13 | 4.99 | 4.50 | 2.00 |
| P. freudenreichii | P 63 | 6.44 | 5.84 | 5.59 | 5.44 |
| P. acidipropionici | EQ42 | 6.07 | 5.91 | 5.68 | 5.19 |
| P. freudenreichii | R 0501 | 5.44 | 2.00 | 0.00 | 0.00 |
| Lb. rhamnosus | R 0011 | 7.08 | 5.13 | 4.70 | 0.00 |
| Lb. casei | R 0256 | 7.83 | 3.70 | 2.00 | 0.00 |
| Propionibacterium | R 1042 | 5.89 | 3.70 | 2.00 | 0.00 |
| Lb. bulgaricus | mibl-2 | 7.66 | 5.65 | 5.10 | 4.80 |
| Lb. rhamnosus | R 1039 | 7.76 | 4.70 | 4.50 | 2.00 |
| Lb. casei | R 0215 | 7.66 | 3.70 | 2.00 | 0.00 |
| Lb. plantarum | HN | 6.60 | 6.20 | 5.04 | 3.70 |
| Lb. plantarum | R 1078 | 6.76 | 6.37 | 4.59 | 2.00 |
| Lb. plantarum | R 0202 | 5.67 | 2.00 | 0.00 | 0.00 |
| Lb. plantarum | R 1027 | 6.49 | 5.35 | 2.00 | 0.00 |
| Lb. acidophilus | LA-HN | 5.94 | 2.00 | 0.00 | 0.00 |
| Lb. acidophilus | BG2F04 | 7.32 | 7.23 | 7.09 | 4.54 |
| Lb. casei paracasei | CRL-431 | 6.62 | 2.00 | 0.00 | 0.00 |
| Bifidobacterium isolate | — | 5.57 | 2.00 | 0.00 | 0.00 |
| S. thermophilus | R 0083 | 6.61 | 2.00 | 0.00 | 0.00 |
| Lb. casei | R 0215 | 6.85 | 2.00 | 0.00 | 0.00 |
| Lb. acidophilus | 150 | 6.30 | 5.37 | 5.15 | 4.91 |
| Lb. delbrueckii spp lactis | R 0187 | 7.54 | 2.00 | 0.00 | 0.00 |
| Lb. acidophilus | R 052 | 6.43 | 5.57 | 5.30 | 5.03 |
| 50S Lb. delbrueckii | R 0187 | 7.16 | 4.39 | 2.00 | 0.00 |
| spp lactis | | | | | |
| 50L Lb. delbrueckii | R 0187 | 6.42 | 2.00 | 0.00 | 0.00 |
| spp lactis | | | | | |

### Example 4

### Compatibility of Bacterial Strains with One Another

Bacterial species and strains of tables 1 to 3 have been tested for their compatibility, mainly based on the potential inhibitory effect of the bacteriocins produced by one strain on other strains.

No inhibitory, neither total nor partial, has been found with any combination of the bacterial strains tested. Therefore, all of the bacterial strains of tables 1 to 3 are suitable to be used as a mixture in a composition.

### Example 5

### Resistance of Bacterial Strains to Gastric Acid and Oxgall

Specific bacterial strains have been evaluated for their resistance to oxgall and gastric acid, either in a dairy composition (D) or in a supplement composition (S) (Table 4). Strains have been noted as being either strongly resistant (✓) or poorly resistant (-) when compared to the performance of other strains from the same species.

TABLE 4
Resistance of specific bacterial strains to oxgall and gastric acid.

| | | | | | Gastric |
|---|---|---|---|---|---|
| | | | Composition | Oxgall | acid |
| Species | Company | Strain | type | resistance | resistance |
| S. thermophilus | HANSEN | TH-3 | S | ✓ | ✓ |
| | HANSEN | Stb01 | D | ✓ | ✓ |
| | HARMONIUM | HA-110 | S | ✓ | ✓ |
| | ROSELL | R-083 | pure strain | ✓ | — |
| P. freudenreichii | ROSELL | R-0501 | S | — | — |
| | HANSEN | PS1 | D | ✓ | ✓ |
| Lb. acidophilus | HANSEN | LA-5 | S | ✓ | — |
| | HANSEN | LA-5 | D | * | * |
| | HARMONIUM | HA-122 | S | ✓ | ✓ |
| | ROSELL | R-052 | S | — | — |
| Lb. casei | HANSEN | CRL-431 | S | ✓ | — |
| | HANSEN | CRL-431 | D | * | * |
| | HANSEN | LC01 | D | — | — |
| Lb. casei | HARMONIUM | HA-108 | S | — | ✓ |
| | ROSELL | R-215 | S | — | ✓ |
| Lb. plantarum | HANSEN | 601387 | S | ✓ | ✓ |
| | HARMONIUM | HA-119 | S | ✓ | — |
| Lb. rhamnosus | HANSEN | LR-35 | D | ✓ | — |
| | HARMONIUM | HA-111 | S | ✓ | ✓ |
| | HARMONIUM | HA-114 | S | ✓ | ✓ |
| | ROSELL | R-011 | S | ✓ | — |
| Lb. bulgaricus | HANSEN | 601383 | S | ✓ | ✓ |
| | HARMONIUM | HA-137 | S | ✓ | ✓ |
| B. lactis | HANSEN | BB12 | S | ✓ | ✓ |
| | HANSEN | BB12 | D | * | * |
| | HARMONIUM | Bb. lactis | S | — | — |
| B. longum | HANSEN | 601377 | S | ✓ | ✓ |
| | HANSEN | BB46 | D | * | * |
| | HARMONIUM | HA-135 | S | ✓ | ✓ |
| | ROSELL | R-175 | S | ✓ | ✓ |
| B. breve | HARMONIUM | HA-129 | S | ✓ | * |
| | ROSELL | R-070 | S | — | * |
| B. infantis | HARMONIUM | HA-116 | S | ✓ | * |
| | ROSELL | R-033 | S | ✓ | * |
| B. bifidum | HARMONIUM | HA-132 | S | ✓ | * |
| | ROSELL | R-071 | S | — | * |

* Test were not performed with those strains

Based on the results of examples 1 to 5, the following bacterial strains have been identified as having the best potential to be used in combination in a probiotic composition: P. freudenreichii R0501, Lb. casei paracasei NV, Lb. plantarum HN, Lb. bulgaricus mibl-2, Lb. acidophilus LA-HN, and Lb. acidophilus BG2F04-2.

### Example 6

### Preparation of the Compositions

The compositions described herein can be produced by various techniques. The following protocol is an example of such a technique, and it will be understood that it is presented here as such. All of the steps prior to mixing were performed by the manufacturer.

Step 1—Bacterial culture: Bacteria are placed in an environment suitable for their growth and proliferation.

Step 2—Fermentation: Bacterial cultures are placed in an environment suitable to allow fermentation. The more proliferation and fermentation occurs, the more the culture media opacifies.

Step 3—Ultrafiltration: Culture media are filtrated to reduce their volumes. Next, specific ingredients are added to the media to ensure the survival of the bacteria for the next step.

Step 4—Lyophilization: The bacterial concentrate is frozen at −40° C. then, by sublimation, ice is transformed in steam, leaving the concentrate as a solid paste, ready to be crushed.

Step 5—Crushing: The solid paste is crushed in order to obtain a fine powder in which the bacteria concentration is very high.

Step 6—Mixing: Powders of different bacterial species, or powder of mixed bacterial species, are mixed together, with the addition or not of various substrates (such as maltodextrin or magnesium stearate) in order to obtain the desired concentration of bacterial species in the resulting mixed powder, according to the composition recipe. Various substrates are added according to the different desired mixes, in concentrations ranging from 3% to 40% of the final composition.

Step 7—Encapsulation: The resulting mixed powder is encapsulated, compressed into tablets or capsules (such as cellulose or other vegetal capsules, or animal gelatin), or bagged, according to the desired form. Traditional techniques known in the art were used for encapsulation.

### Example 7

### Formulation of BIACTIVE JUNIOR™

The following formulation serves as an example of the composition described herein. BIACTIVE JUNIOR™ is a probiotic formula for the prevention and treatment of gastroenteric disorders-related symptoms. Those disorders include, without being limited to, disturbances in gastric flora, viral and bacterial gastroenteritis, antibiotic-related diarrhea and lactose-intolerance-related diarrhea. Furthermore, BIACTIVE JUNIOR™ can be administered in a prophylactic manner when there is a risk of contracting viral or bacterial gastroenteritis. During an antibiotic treatment BIACTIVE JUNIOR™ can have beneficial effects in order to prevent diarrhea caused by gastric flora disturbance.

Typically, a package of BIACTIVE JUNIOR™ contains 30 capsules, each capsule containing 17 billions bacteria from 9 bacterial species associated with 4 bacterial genus. The bacterial species included in BIACTIVE JUNIOR™ are Lactobacillus acidophilus, Lactobacillus paracasei, Propionibacterium freudenreichii, Bifidobacterium lactis, Bifidobacterium bifidum, Bifidobacterium breve, Bifidobacterium infantis, Bifidobacterium longum, and Streptococcus thermophilus. Non-medicinal ingredients include natural lyophilized grape juice, microcrystalline cellulose, maltodextrin, magnesium stearate, citric acid and silica gel.

BIACTIVE JUNIOR™ is formulated for children 2 to 6 years old (1 capsule per day) and children 7 to 12 years old (1 to 2 capsules per day). It is designed to be ingested at meal time in order to maximize product efficacy. Alternatively, capsules can be mixed with cold food (e.g. jam, yogurt, water, juice, etc). Posology is also adapted with regards to the disorder in need of a treatment. For example, for antibiotic-induced diarrhea, the recommended dose is to be taken for all the antibiotic treatment duration, and for the next five days following the antibiotic treatment.

In order to present a probiotic effect and to reach the targeted area of action, i.e. the colon, a bacteria needs to resist to the various physiological barriers present in the gastrointestinal tract, including the gastric acidity of the stomach and biliary salts. The bacterial species present in BIACTIVE JUNIOR™ have all been tested in vitro for their resistance to those barriers. A dynamic stimulator of the gastrointestinal system has been used to estimate the survival rate and the metabolic stability of the bacterial species selected.

Bacterial species used in BIACTIVE JUNIOR™ are selected and their concentrations and relative proportions to one another are carefully calculated and tested in order to produce an optimal and synergic effect on gastrointestinal flora of children from 2 to 12 years old. In vitro tests as well as clinical studies in human have been performed in order to finalize the formulation.

TABLE 5
Formulation for BIACTIVE JUNIOR ™

| Bacterial species | Strain | Range | Percentage | Quantity per dose |
|---|---|---|---|---|
| Lb. acidophilus | LA-5 | 10-40% | 15% | 1.50E+09 |
| Lb. casei | CRL-431 | 5-30% | 15% | 1.50E+09 |
| B. lactis | BB-12 | 5-40% | 15% | 1.50E+09 |
| B. longum | HA-135 | 5-30% | 10% | 1.00E+09 |
| B. bifidum | HA-132 | 5-20% | 10% | 1.00E+09 |
| B. breve | HA-129 | 5-20% | 10% | 1.00E+09 |
| B. infantis | Chr | 5-30% | 10% | 1.00E+09 |
| P. freudenreichii | R-0501 | 5-30% | 10% | 1.00E+09 |
| S. thermophilus | HA-110 | 2-20% | 5% | 5.00E+08 |
| Total bacteria | | | | 1.00E+10 |

Storage of BIACTIVE JUNIOR™ is recommended at a temperature of about 4° C. (about 2° C. to 6° C.), but it can also be frozen. At those conditions, BIACTIVE JUNIOR™ is stable for a period of 2 years, if kept in original container.

### Example 8

### Formulation of BIACTIVE TOURISTA™

The following formulation serves as an example of the composition described herein. BIACTIVE TOURISTA™ is a probiotic formula for reconstituting, stabilizing or normalizing the gastric flora, and for the prevention of traveler's diarrhea as well as alleviating symptoms thereof (diarrhea, vomiting, nausea, fever, abdominal pain).

Typically, a package of BIACTIVE TOURISTA™ contains 30 capsules, each capsule containing 31 billions bacteria from 8 bacterial species associated with 4 bacterial genus. The bacterial species included in BIACTIVE TOURISTA™ are Lactobacillus plantarum, Lactobacillus rhamnosus, Lactobacillus acidophilus, Lactobacillus paracasei, Propionibacterium freudenreichii, Bifidobacterium bifidum, Bifidobacterium breve, and Streptococcus thermophilus. Non-medicinal ingredients include vegetal capsule, maltodextrin, magnesium stearate, and silica gel.

Beside testing for their survival and stability to GI stresses, strains for BIACTIVE-TOURISTA™ have been selected based on two additional tests:
Testing for stability at room temperature. Since this product is specifically designed to be used during trips and vacations to warmer countries, there is a high risk that the product be kept at warmer temperature than 4° C. Bacterial strains that were stable at room temperature have been selected.
Clinical testing for the efficiency of each strain to decrease, prevent and stop the symptoms of traveler's diarrhea. Bacterial strains that presented strong clinical data for their efficiency but were more or less stable at 37° C. have been overdosed in the composition.

BIACTIVE TOURISTA™ is formulated for children from 2 to 12 years old (½ to 1 capsule per day) and or children of more than 12 years old and adults (1 to 2 capsules per day). The recommended posology involves the taking of the recommended dosage 2 days prior to the travel, and for the next 5 days following the end of the travel. It is designed to be ingested at meal time in order to maximize product efficacy. Alternatively, capsules can be mixed with cold food (e.g. jam, yogurt, water, juice, etc).

In order to present a probiotic effect and to reach the targeted area of action, i.e. the colon, a bacteria needs to resist to the various physiological barriers present in the gastrointestinal tract, including the gastric acidity of the stomach and biliary salts. The bacterial species present in BIACTIVE TOURISTA™ have all been tested in vitro for their resistance to those barriers. A dynamic stimulator of the gastrointestinal system has been used to estimate the survival rate and the metabolic stability of the bacterial species selected.

BIACTIVE TOURISTA™ is formulated to prevent infections caused by pathogens usually found in contaminated food and water, to alleviate symptoms associated with traveler's diarrhea, as well as to facilitate general gastrointestinal health.

Bacterial species used in BIACTIVE TOURISTA™ are selected and their concentrations and relative proportions to one another are carefully calculated and tested in order to produce an optimal and synergic effect on gastrointestinal flora. In vitro tests as well as clinical studies in human have been performed in order to finalize the formulation.

Storage of BIACTIVE TOURISTA™ is recommended at a temperature of about 4° C. (about 2° C. to 6° C.), but it can also be frozen. At those conditions, BIACTIVE TOURISTA™ is stable for a period of 2 years, if kept in original container.

TABLE 6
Formulation for BIACTIVE TOURISTA ™

| Bacterial species | Strain | Range | Percentage | Quantity per dose |
|---|---|---|---|---|
| Lb. rhamnosus | HA-114 | 10-30% | 20% | 4.00E+09 |
| Lb. acidophilus | LA-5 | 10-30% | 20% | 4.00E+09 |
| Lb. casei | CRL-431 | 10-30% | 15% | 3.00E+09 |
| Lb. plantarum | 601387 | 10-30% | 15% | 3.00E+09 |
| B. bifidum | HA-132 | 5-20% | 10% | 2.00E+09 |
| B. breve | HA-129 | 5-20% | 10% | 2.00E+09 |
| S. thermophilus | HA-110 | 5-15% | 5% | 1.00E+09 |
| P. freudenreichii | R-0501 | 5-20% | 5% | 1.00E+09 |
| Total bacteria | | | | 2.00E+10 |

### Example 9

### Formulation of BIACTIVE 12™

The following formulation serves as an example of the composition described herein. BIACTIVE 12™ is a probiotic formula for the prevention and treatment of gastroenteric disorders-related symptoms. Those disorders include, without being limited to, disturbances in gastric flora, irritable bowel syndrome-related symptoms (diarrhea, colic, distension, constipation), viral and bacterial gastroenteritis, and antibiotic-related diarrhea. Furthermore, BIACTIVE 12™ can be administered jointly with a treatment for inflammatory diseases of the intestine (such as Crohn's disease or ulcerative colitis), C. difficile-induced diarrhea, H. pylori-related gastric ulcer, etc.

Typically, a package of BIACTIVE 12™ contains 40 capsules, each capsule containing 31 billions bacteria from 12 bacterial species associated with 4 bacterial genus. The bacterial species included in BIACTIVE 12™ are Lactobacillus plantarum, Lactobacillus rhamnosus, Lactobacillus acidophilus, Lactobacillus paracasei, Propionibacterium freudenreichii, Bifidobacterium lactis, Bifidobacterium bifidum, Bifidobacterium breve, Bifidobacterium infantis, Bifidobacterium longum, Lactobacillus bulgaricus, and Streptococcus thermophilus. Non-medicinal ingredients include vegetal capsule, maltodextrin, magnesium stearate, and silica gel.

BIACTIVE 12™ is formulated for adults and children over 12 years old. It is designed to be ingested at meal time in order to maximize product efficacy. Alternatively, capsules can be mixed with cold food (e.g. jam, yogurt, water, juice, etc). Posology is also adapted with regards to the disorder in need of a treatment. For example, 1 to 3 capsules per day, in accordance with symptoms intensity, are recommended for the treatment of irritable bowel disease, gastroenteritis, Crohn's disease and ulcerative colitis. For antibiotic-induced diarrhea, 1 to 3 capsules per day are recommended 3 hours after the antibiotic intake, during all of the antibiotic treatment and up to 5 days following the end of the treatment. For C. difficile infections, 3 capsules per day are recommended.

In order to present a probiotic effect and to reach the targeted area of action, i.e. the colon, a bacteria needs to resist to the various physiological barriers present in the gastrointestinal tract, including the gastric acidity of the stomach and biliary salts. The bacterial species present in BIACTIVE 12™ have all been tested in vitro for their resistance to those barriers. A dynamic stimulator of the gastrointestinal system has been used to estimate the survival rate and the metabolic stability of the bacterial species selected.

BIACTIVE 12™ is formulated to act on physiological disorders associated with irritable bowel syndrome, gastric and intestinal distension, modification of intestinal transit (constipation and diarrhea), digestive disorders, nutriment absorption disorders and intestinal inflammation. BIACTIVE 12™ induces cell regeneration in intestinal mucosa.

Bacterial species used in BIACTIVE 12™ are selected and their concentrations and relative proportions to one another are carefully calculated and tested in order to produce an optimal and synergic effect on gastrointestinal flora. In vitro tests as well as clinical studies in human have been performed in order to finalize the formulation.

TABLE 7
Formulation for BIACTIVE 12 ™

| Bacterial species | Range | Percentage | Quantity per dose |
|---|---|---|---|
| Lb. rhamnosus | 10-30% | 18% | 3.60E+09 |
| Lb. plantarum | 10-30% | 18% | 3.60E+09 |
| Lb. acidophilus | 10-30% | 10% | 2.00E+09 |
| Lb. casei | 5-20% | 10% | 2.00E+09 |
| P. freudenreichii | 5-20% | 10% | 2.00E+09 |
| B. lactis | 5-20% | 10% | 2.00E+09 |
| B. bifidum | 5-10% | 5% | 1.00E+09 |
| B. breve | 5-10% | 5% | 1.00E+09 |
| B. infantis | 5-15% | 5% | 1.00E+09 |
| B. longum | 3-10% | 4% | 8.00E+08 |
| Lb. bulgaricus | 2-10% | 2.5% | 5.00E+08 |
| S. thermophilus | 2-10% | 2.5% | 5.00E+08 |
| Total bacteria | | | 2.00E+10 |

Storage of BIACTIVE 12™ is recommended at a temperature of about 4° C. (about 2° C. to 6° C.), but it can also be frozen. At those conditions, BIACTIVE 12™ is stable for a period of 2 years, if kept in original container.

### Example 10

### Capacity of BIACTIVE 12™ Species to Survive Gastro-Intestinal (GI) Tract Passage

Capacity of probiotic bacteria to survive to GI tract passage is a determining factor in evaluating their probiotic potential. This capacity is generally evaluated on populations that have been successively exposed to acid and gastric stresses in a static model. Simulating different physiological conditions found in vivo during digestion, the gastro-intestinal dynamic model TIM-1 (TNO Nutrition and Food Research Institute, Zeist, The Netherlands) has been used to evaluate the bacterial strains present in BIACTIVE 12™.

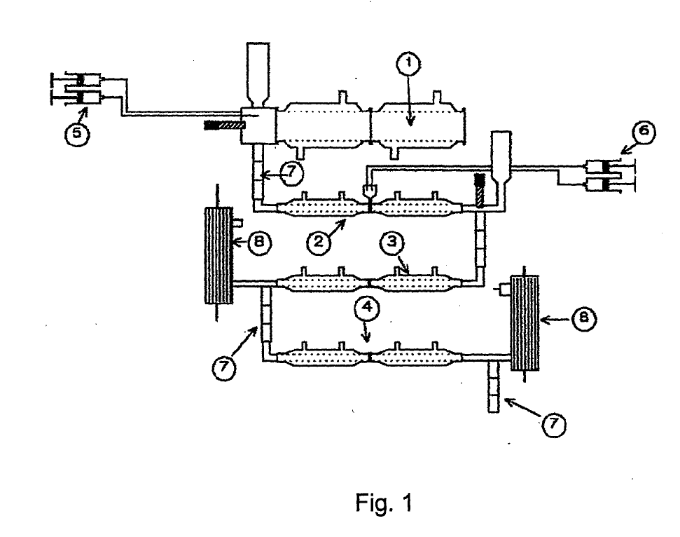

The TIM-1 apparatus (FIG. 1) is composed of four consecutive sections simulating the stomach, duodenum, jejunum and ileum, connected with computer-controlled mechanical valves. Peristalsis and gastric emptying are simulated with flexible tubing. Synthetic secretions simulating saliva, gastric acid, biliary and pancreatic secretions are introduced in the different sections by computer-controlled pumps. The jejunum and ileum are equipped with hollow fibers allowing chyme dialysis. Levels of pH and temperature are controlled by probes connected to the computer. It has been shown that this apparatus reliably and efficiently simulates physiological conditions (Marteau, 1997).

The apparatus was sterilized overnight with a chloride solution prior to use. Many washings with deionized water were performed on the apparatus in order to remove all of the chloride solution before experimentation was started. Washing efficiency was monitored by measuring pH of deionized water after washing. Sterility was assessed by microbial counting of the total aerobic flora, yeasts, molds and coliform flora according to the Petrifilm™ method (3M, St-Paul, Minn.).

Experimentation was performed with the BIACTIVE 12™ supplement (containing 12 bacterial strains from 4 different species). Since the intake of dairy product is usually recommended with probiotic supplements ingestion, a capsule of BIACTIVE 12™ was mixed in 300 ml of microfiltrated milk (Pur Filtre™, Lactantia, Parmalat) before being digested for 6 hours in TIM-1 apparatus. Microbiological samples of 1 ml were taken in each sections every 30 or 60 minutes, throughout the 6 hour digestion. Evaluation of the microbial content of the milk (<1 colony-forming units (CFU)/ml) was performed according to the Petrifilm™ method (3M).

Sample were kept on ice and put on Petri dishes within one hour. Total bifidobacteria and lactobacilli populations were quantified according to the drop plate technique with two distinct media, thus allowing the differentiation of bacterial species. Culture media were incubated according to the required parameters for each bacterial species. Results are expressed in log CFU representative of the total bacterial number calculated from the mean of two assays (table 8).

TABLE 8
Population of bifidobacteria and lactobacilli during GI transit.

| | | Lactobacillus | Lactobacillus | Bifidobacteria | Bifidobacteria |
|---|---|---|---|---|---|
| Section | Time | CFU (log) | standard error | CFU (log) | standard error |
| Stomach | 0 | 9.6 | 0.0 | 9.4 | 0.1 |
| | 30 | 8.8 | 0.2 | 8.8 | 0.4 |
| | 60 | * | 0.0 | 6.2 | 0.0 |
| | 90 | * | 0.0 | * | 0.0 |
| Duodenum | 30 | 8.3 | 0.2 | 8.3 | 0.4 |
| | 60 | 6.1 | 0.0 | * | 0.0 |
| | 90 | 6.5 | 0.0 | 6.2 | 0.0 |
| | 120 | 6.1 | 0.0 | 6.3 | 0.0 |
| Jejunum | 60 | 8.0 | 0.3 | 8.3 | 0.2 |
| | 90 | 7.5 | 0.1 | 7.9 | 0.2 |
| | 120 | 7.3 | 0.3 | 7.5 | 0.3 |
| | 180 | 7.4 | 0.0 | 6.9 | 0.0 |
| Ileum | 90 | 7.9 | 0.1 | 8.1 | 0.0 |
| | 120 | 7.7 | 0.2 | 7.8 | 0.2 |
| | 180 | 7.4 | 0.2 | 7.7 | 0.3 |
| | 240 | 7.5 | 0.2 | 7.8 | 0.1 |
| | 300 | 7.2 | 0.4 | 7.7 | 0.1 |
| | 360 | 7.4 | 0.1 | 7.8 | 0.1 |

* Populations present in sample were below detection level (6 log).

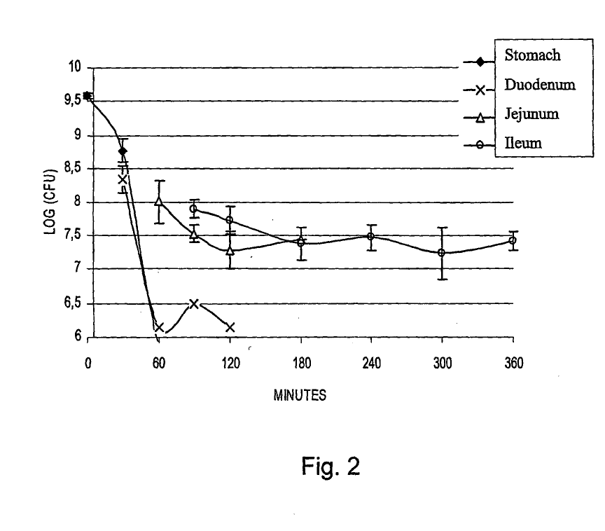

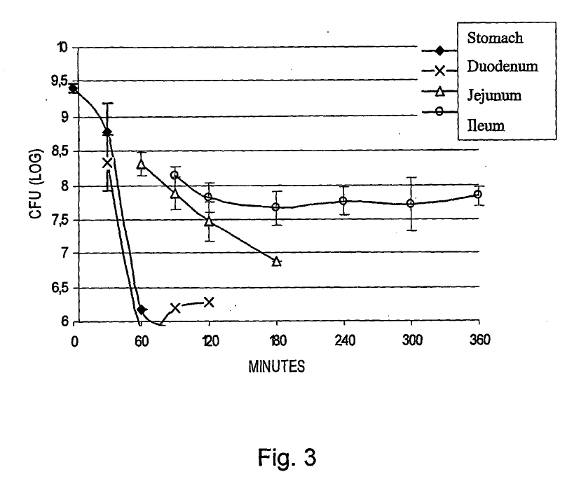

FIGS. 2 and 3 respectively show the populations of lactobacilli and bifidobacteria during the GI transit. Along with table 8, those figures show a very good survival rate of lactobacilli and bifidobacteria during GI transit.

Initial ingested populations, represented by time=0 in the stomach, were estimated at 3.9×10⁹ CFU lactobacilli (9.6 log) and 2.5×10⁹ CFU for bifidobacteria (9.4 log). By comparison, a BIACTIVE 12™ capsule respectively contains 10×10⁹ CFU (10 log) and 13×10⁹ CFU (10.1 log) of those bacteria. The difference has been attributed to the presence of milk components (proteins, fat) in the stomach sample, the temperature, the rehydration media and the microbiological measure error. In the course of the 6 hours digestion, a loss of 2.2 and 1.6 log has been observed respectively for lactobacilli and bifidobacteria, indicating that the number of those bacteria in the small intestine after 6 hours (respectively of 2.9 and 6.8×10⁷ CFU) was still sufficient for a therapeutic effect.

A notable decrease was observed in both populations after 60-90 minutes in the stomach. This decrease was caused by the extremely low pH values found in the stomach during this period.

After the 6 hour digestion, the population of bifidobacteria was slightly superior to that of lactobacilli. Tolerance of lactobacilli and bifidobacteria to the various stresses encountered during GI transit has been found to vary on the strain tested. Variations ranging from 0.5 to 4 log have been observed in various strains of the same species (Berrada et al., 1991).

### Example 11

### Stability of BIACTIVE TOURISTA™, BIACTIVE 12™, and BIACTIVE JUNIOR™ Under Various Storing Conditions

While no study has yet established the minimal efficient dosage for a given strain, the populations comprised in a probiotic supplement must be sufficiently high for the subject to obtain a therapeutic effect. It is generally accepted that daily dosages of about 10⁹-10¹¹ CFU are associated with appreciable therapeutic effects. A high initial number of bacteria should therefore compensate for the loss observed during GI transit. BIACTIVE products are mainly composed of lyophilized probiotic cultures and their initial dosage ensure the presence of many billions of living bacteria in each capsule.

While their metabolic activity is maintained at a very low level, those lyophilized bacteria are not totally inert, and their respiration never completely stops. Bacterial stability toward lyophilization treatment is dependent on the strain. Once encapsulated, probiotic lyophilized bacteria survival is largely dependent on the storage conditions. Storage temperature, wrapping conditions, water activity (A_{w}), relative humidity, additives and oxygen presence are factors affecting bacterial survival (Champagne et al. 1991). Regarding oxygen, it has been shown that the viability of bifidobacteria strains present in a probiotic yogurt were increase when a glass container was used, rather than a plastic one, oxygen being deleterious to bifidobacteria (Shah, 2000).

Storage temperature also plays a major role for bacteria survival. Some authors have noted that the viability of a given strain could be diminished by a factor of 100 when stored at 20° C. instead of 4° C. (Champagne et al. 1991). The relationship between storage temperature and bacterial survival or bacterial death has been confirmed by many additional authors since.

In the present study, three types of supplements were tested (BIACTIVE TOURISTA™, BIACTIVE JUNIOR™ and BIACTIVE 12™) for storage in bottles at 4° C.+/−1° C. (refrigerator), 10° C.+/−1° C. (refrigerator), 25° C.+/−1° C. (room temperature). Additional testing was performed on bottles of BIACTIVE TOURISTA™ for 27° C.+/−1° C. (incubator) and 37° C.+/−1° C. (incubator). A temperature of 4° C. represent the usual recommended storage temperature for lyophilized probiotic supplement in order to ensure a maximum stability of the powders. A temperature of 10° C. represent the potential temperature variation of a commercial refrigerator, such as the ones used by drug stores. A temperature of 25° C. reflect a room temperature storage. Temperatures of 27° C. and 37° C. represent extreme storage conditions such the ones encountered in countries having warmer climates (during vacations for example), or in a non-refrigerated delivery truck in the summer time.

Each supplement bottle was stored at those temperatures for periods of 7, 14, 21, 28 or 48 days (table 9). Three capsules from each bottle were tested at the end of the storage periods for counting of lactobacilli and bifidobacteria. Streptococcus thermophilus et Propionibacterium freudenreichii, present in all supplements, were not counted. Results show the combined populations of lactobacilli and bifidobacteria in the supplements. The supplement bottles were initially stored at 4° C. for 4 months prior to testing.

TABLE 9
Sampling schedule

Days of sampling

| Temperatures | 0* | 7 | 14 | 21 | 28 | 48 |
|---|---|---|---|---|---|---|
| 4° C. | X | | | | | |
| 10° C. | | X | | X | | |
| 25° C. | | X | X | X | X | X |
| 27° C. | | X | X | X | | |
| 37° C. | | X | X | X | | |

*Samples stored for 4 months at 4° C.

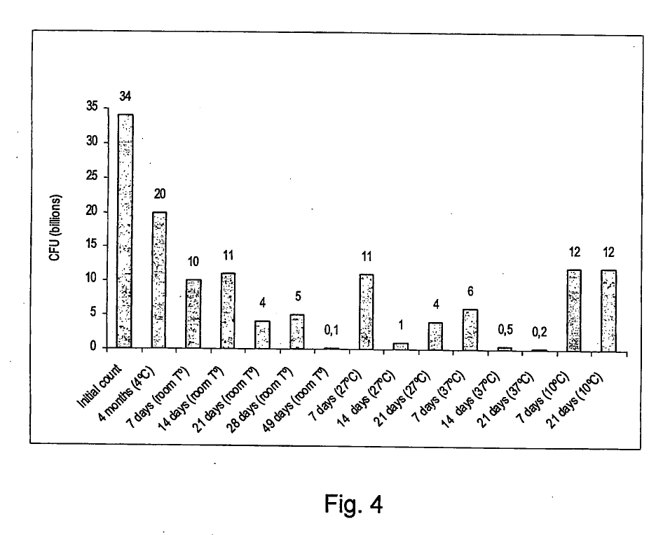

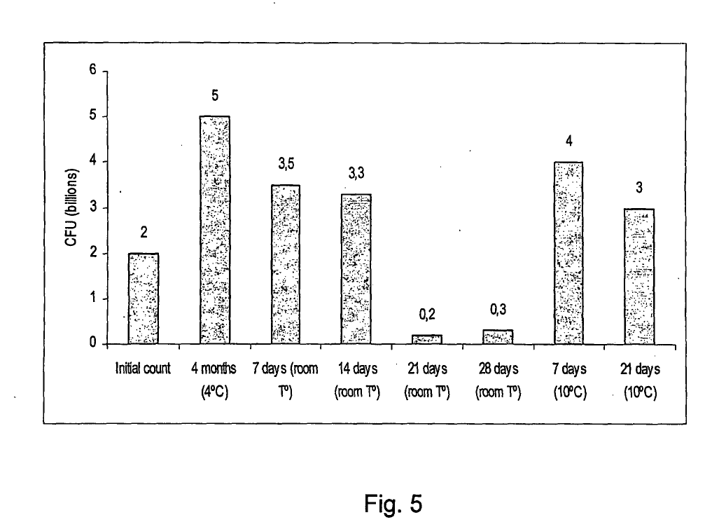

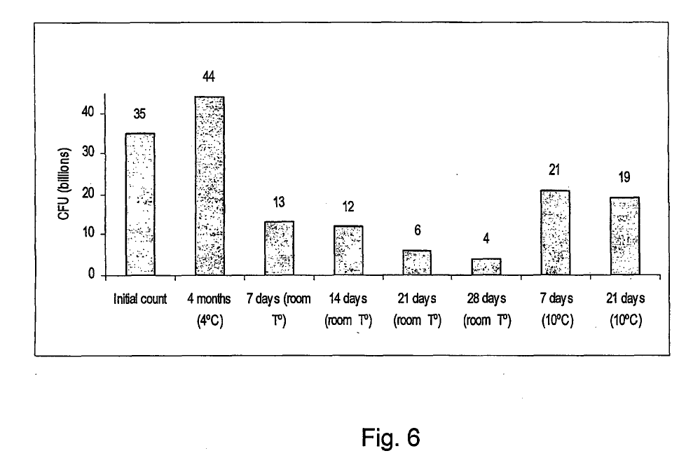

Storage temperature had a major effect on the maintenance of probiotic bacteria viability in the supplements. Table 10, along with FIGS. 4, 5 and 6, show the results of this study.

TABLE 10
Stability of supplements over time

| | Storage | | CFU | CFU | Loss |
|---|---|---|---|---|---|
| Supplement | temperature | Days | (billions) | (log) | (log) |
| BIACTIVE | 4° C. | 0 | 34 | 10.5 | |
| TOURISTA ™ | | 120 | 20 | 10.3 | 0.2 |
| | 10° C. | 7 | 12 | 10.1 | 0.4 |
| | | 21 | 12 | 10.1 | 0 |
| | 25° C. | 7 | 10 | 10 | 0.5 |
| | | 14 | 11 | 10 | 0.5 |
| | | 21 | 4 | 9.6 | 0.9 |
| | | 28 | 5 | 9.7 | 0.8 |
| | | 49 | 0.1 | 8 | 2.5 |
| | 27° C. | 7 | 11 | 10 | 0.5 |
| | | 14 | 1 | 9 | 1.5 |
| | | 21 | 4 | 9.5 | 1.0 |
| | 37° C. | 7 | 6 | 9.8 | 0.7 |
| | | 14 | 0.5 | 8.7 | 1.8 |
| | | 21 | 0.2 | 8.2 | 2.3 |
| BIACTIVE | 4° C. | 0 | 2* | 9.3 | — |
| JUNIOR ™* | | 120 | 5 | 9.7 | — |
| | 25° C. | 7 | 3.5 | 9.5 | — |
| | | 14 | 3.3 | 9.5 | — |
| | | 21 | 0.2 | 8.3 | — |
| | | 28 | 0.3 | 8.5 | — |
| | 10° C. | 7 | 4 | 9.6 | — |
| | | 21 | 3.2 | 9.5 | — |
| BIACTIVE | 4° C. | 0 | 35 | 10.5 | |
| 12 ™ | | 120 | 44 | 10.6 | 0 |
| | 25° C. | 7 | 13 | 10.1 | 0.4 |
| | | 14 | 12 | 10.1 | 0.4 |
| | | 21 | 6 | 9.8 | 0.7 |
| | | 28 | 5 | 9.7 | 0.8 |
| | 10° C. | 7 | 21 | 10.3 | 0.2 |
| | | 21 | 19 | 10.3 | 0.2 |

*Methodology used for the initial counting of this product has probably underestimated the present populations.

Important population losses were noted with all supplement stored above 4° C. Viability was particularly affected over 10° C. The samples stored at 4° C. presented the expected population loss for this kind of product. However, the population losses observed were in the reasonably acceptable range of 0.1 to 0.2 log CPU for a four month period.

One should note at this point that the manufacturer's recommendations for this kind of product address only product kept in their original wrapping, impermeable to oxygen, and that the usual recommended storage temperature is under 4° C. Moreover, it had been demonstrated by Ishibashi et al. (1985) that the stability of lactic bacteria was more easily preserved at A_{w} values of 0.1 to 0.2.

Those results show the importance of preserving cool temperatures during storage, and the conservation of the product at 4° C. or less by the user. Storage at room temperature should not be considered for periods exceeding 7 days. Bacterial counting of non-sealed samples stored at room temperature revealed the importance of preserving an atmosphere having low humidity and oxygen levels for bacterial survival.

### Example 12

### Incorporation of Bacterial Probiotics to a Beverage

The development of a fermented milk has been considered (skim cow milk), which would combine the effects of probiotic strains with nutraceutical molecules. The lactose present in the product could be used as an energy source by the bacteria, which could be monitored by measuring sugar traces once fermentation is complete.

By doing so, the bacterial multiplication would be favored, which would provide the advantages of:
Decreased cost related to lesser bacterial inoculation;
Development of organoleptic traits caused by the fermentation;
Attainment of an elevated final number of bacteria, increasing the probiotic potential.

Contrary to the available probiotic supplement commercially available, usually containing 1 or 2 species, we looked to making a supplement containing a minimum of 7 bacterial strains from 4 different bacterial species, having a concentration ranging from 250 billions bacteria at manufacture time to 50 billions bacteria at usage time, even after up to 60 days of storage at 4° C.

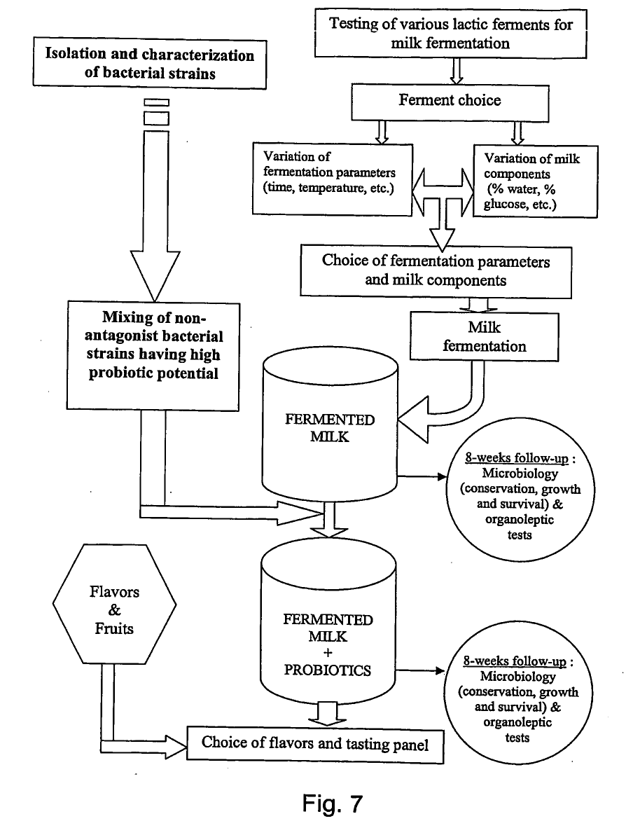

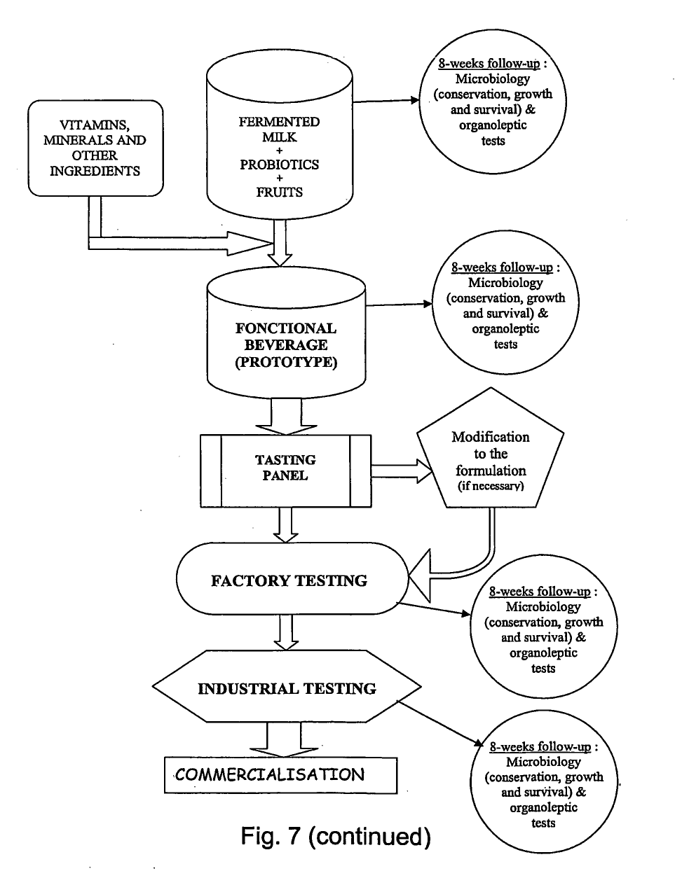

The expected problems related to the making of such a supplement were 1) the optimal growth of all the strains to obtain a total number of bacteria reaching 50 billions after 60 days of storage at 4° C., while keeping a balance between the different strains; 2) lactose degradation; 3) the presence of 4 different species of bacteria, each with their own restrictions and particularities; and 4) the organoleptic traits of the final product (taste, texture, acidity, etc). The development of the beverage was performed according to the scheme of FIG. 7.

The first step was the fermentation of the milk with each of the strains. Individual fermentation with one strain was first performed, followed by pairing of strains, and fermentation with multiple strains. Strains from different commercial sources were tested this way for their growth and the organoleptic traits they gave to the final product. Finally, a mixture containing the 7 most interesting strains was tested, based on the following criteria:
Inoculation values: Bifidobacteria at an initial concentration above 5×10⁷ CFU/ml gave a sour taste to the final product. Dosage was established so as to give the highest number of bifidobacteria after fermentation, without reaching the critical value of 5×10⁷ CFU/ml. Streptococcus acidophilus had to be repeatedly tested so as to start the medium acidification without taking over the other strains. Lb. casei and Lb. acidophilus had also to be tested thoroughly so as to obtain high numbers of bacteria without sacrificing the taste of the final product.
Fermentation length: In order to increase the total number of bacteria in the final product (particularly bifidobacteria and lactobacilli), we had to increase the length of the fermentation in order for the bacteria to develop to their full potential without lowering the final pH. We achieved that by lowering the inoculation value of the “fermentation starter”, S. thermophilus, and by lowering the fermentation temperature.
pH: Strain survival in the final product was improved by maintaining a pH over 4.2 for the duration of the storage. This was established by performing different fermentations that were stopped at various pH values. A fragile equilibrium had to be reached at pH 4.2 since at higher values, availability of residual lactose was affected.
Total solid concentration: Various concentrations of total solids were tested in order to determine their influence on bacterial survival and on the texture of the final product.

Bacterial counts of over 230 billions bacteria per portion were obtained in the course of those experiments, indicating that values of 20 billions after 60 days of storage at 4° C. was possible.

More than 200 fermentations with different inoculation values of different strains have been performed in order to achieve the various ratios between the bacterial strains in the compositions presented herein. An antagonistic effect was observed in fermented milk between different bacterial strains. Balance was found between all the bacterial strains after multiple tests.

The organoleptic characteristics of the final products were also indicative of a product that was appealing to the user. Moreover, those organoleptic characteristics can be enhanced with additional ingredients such as, for example, fruit extracts and natural sugars.

The addition of prebiotics such as inuline and oligofructose was also tested during fermentation in order to allow for a better survival of bacterial strains during storage. Positive effect of probiotics on probiotics survival in the gut was noted.

### Example 13

### Incorporation of Selected Bacterial Strains to a Soy Milk-Based Beverage

This was performed in order to provide a product that is acceptable to lactose-intolerant users and to users wanting non-dairy products. The mixture was the same as the one used for skim milk, but since that mixture contains lactic bacteria, documented for their growth in dairy product, additional testing had to be performed. Conditions similar to those giving the best results for skim milk were first tested.

Results obtained with regards to organoleptic properties of the final product as well as probiotic bacteria growth (50 billions bacteria after 60 days) were interesting. Even if lactose present in milk can be used as an energy source for the bacteria, analysis showed that bacterial survival for higher in soy milk, despite the fact that no lactose is present in soy milk.

### Example 14

### Microbial Counting

In order to ensure a high number of probiotic bacteria in the final product, an adequate counting method had to be used. Since many different species were comprised in the product, isolating those strains required differential media, allowing the growth of the bacteria of interest while limiting that of others. The availability of information in literature or from manufacturers was not sufficient, mostly based on the concentration found in the final product and the fact that several species and strains were present.

Selective media had to be elaborated and tested for each of the bacterial species (lactobacilli, bifidobacteria, propionibacteria and streptococcus). Four media were identified that allowed for the counting of six out of the seven strains comprised in the fermented milk. One for S. thermophilus, one for the bifidobacteria (B. lactis and B. longum together), one for Lb. plantarum and Lb. casei, and one for Lb. acidophilus. Propionibacteria were not counted for a lack of an adequate medium.

### Example 15

### Elaboration of the Composition of a Functional Beverage

This study was performed in order to develop appealing tastes for the final product. Knowing that some fruits and other aliments can inhibit bacteria (because, amongst other things, of their acidity and enzymatic activity), the added ingredients needed to be compatible with probiotics, i.e. preserving the therapeutic properties while ensure bacterial survival over time. Moreover, additional characteristics of the added ingredients were identified as being undesirable, such as artificial flavors, conservation agents and stabilizing agents (thickening agents and the likes).

The following mixes were prepared to undergo pasteurization at 95° C. for 5 minutes:
1) 65 g unsweetened mango puree (Delifruit™)+31.4 g frozen apple juice concentrate (Kerr 70° Brix™)+52 g running water;
2) 61 g unsweetened apple puree (Delifruit™)+39.3 g frozen apple juice concentrate (Kerr 70° Brix™)+46.3 g running water;
3) 65 g unsweetened mango puree (Delifruit™)+31.7 g frozen mango juice concentrate (Kerr 70° Brix™)+52 g running water;
4) 62.4 g unsweetened peach puree (Delifruit™)+30.8 g frozen apple juice concentrate (Kerr 70° Brix™)+45.2 g running water;

Mixing was performed my magnetic agitation in beakers on hot plates. A 0.1 ml sample of each mix was tested in duplicate for fecal coliforms (according to the Violet Red Bile Agar method), yeasts and mold (according to the Potato Dextrose Agar method) after pasteurization. Results from these tests are presented in table 11.

TABLE 11
Microbial analysis of fruit mixes

| Mix | Total coliforms | Total coliforms | Yeasts and molds | Yeasts and molds |
|---|---|---|---|---|
| number | (pre-pasteurization) | (post-pasteurization) | (pre-pasteurization) | (post-pasteurization) |
| 1 | 0 | 0 | 0 | 0 |
| 2 | 0 | 0 | 0 | 0 |
| 3 | 0 | 0 | 1 | 0 |
| 4 | 0 | 0 | 5 yeasts + 1 mold | 0 |

The pasteurization process can be validated for yeasts and mold since yeasts and molds present in mix 4 have been eliminated in the post-pasteurization sample. However, the process could not be validated for fecal coliforms since none were present pre-pasteurization.

Further testing were performed with additional ingredients, such as coconut and blue berry. Natural flavors were also tested, but the resulting products were not in line with the desired organoleptic properties, i.e. the final tastes were reminiscent of artificial flavors, and taste was persistent in mouth after consumption.

### Example 16

### Preliminary Study on the Production of Fermented Milk

The fermentation technique was elaborated based on manufacturer's recommendations with the R-0052 strain of Lb. acidophilus (150×10⁹ CFU/g). All materials was washed and disinfected prior to use.

Twelve percent reconstituted milk was prepared by mixing 142 g of powder skim milk with 1041 g of spring water (Selection Merite™), for a total volume of about 1200 ml. Milk was then pasteurized at 93° C. for 30 minutes.

An inoculation value of 0.1% was used by adding 1.2 g of Lb. acidophilus R-0052 to the 1183 g of milk at 38.5° C. for a total of 1.5×10⁸ CFU/g. Starting pH was of 6.38. Fermentation was carried at 37° C. for 19 hours.

The resulting fermented milk presented a coagulated appearance with syneresis and had a firm gel texture. It had a good taste despite a strong acid flavor. After manual homogenization with a whip, texture was still very thick. The pH was of 4.04.

Fermented milk was too thick for the desired results and pH was too low. Reducing the length of fermentation would allow for a higher pH value. In order to reduce acidity of the fermented milk, fermentation must be closely monitored to follow pH evolution. Fermentation should however not be stop before milk coagulation (pH of 5.20).

### Example 17

### Preparation of Fermented Milk with a Mix of Selected Bacterial Strains

Based on previous tests, fermentation of a 14% reconstituted skim milk (from powder) with a ferment composed of a mix of selected bacterial strains was performed. Relationship between fermentation time/fermentation temperature was evaluated in order to achieve a final pH of about 4.30+/−0.10 in the resulting fermented milk, with predetermined inoculation values. Bacterial strains used were the following: S. thermophilus (R-0083, 202×10⁹ CFU/g); Lb. delbreuckii ssp. bulgaricus (601383, 50×10⁹ CFU/g); Lb. acidophilus (R-0052, 150×10⁹ CFU/g); Lb. casei (601379, 100×10⁹ CFU/g); B. longum (601377, 100×10⁹ CFU/g); B. lactis (601373, 100×10⁹ CFU/g); and P. freudenreichii (P63, 9×10⁹ CFU/g).

A first fermentation was performed at 37° C. for 8 hours, and a second fermentation was performed at 42° C. for 6 hours. Both fermentations were performed in duplicates. Formulations used in 800 g of reconstituted skim milk are presented in table 12:

TABLE 12
Formulations used for testing relationship
between fermentation temperature and time

| | | |
|---|---|---|
| Species | Inoculation value | Inoculation weight |

| | | | |
|---|---|---|---|
| S. thermophilus | 1 × 10⁷ | CFU/g | 0.040 g |
| Lb. delbreuckii ssp. | 0.33 × 10⁷ | CFU/g | 0.051 |
| Lb. acidophilus | 0.33 × 10⁷ | CFU/g | 0.018 |
| Lb. casei | 0.33 × 10⁷ | CFU/g | 0.026 |
| B. longum | 1 × 10⁷ | CFU/g | 0.080 |
| B. lactis | 1 × 10⁷ | CFU/g | 0.080 |
| P. freudenreichii | 1 × 10⁵ | CFU/g | 0.009 |

Final pH of the fermented milks were measured. Taste was evaluated by a degustation panel. Results are presented in table 13.

TABLE 13
pH and appreciation of taste of fermented milks

| 37° C. for 8 hours | 42° C. for 6 hours |
|---|---|
| pH: 4.42 | pH: 4.25 |
| Very good taste, | Less interesting taste |
| no powder tasting. | than that of 37° C. |
| Could be more acidic. | More bitter than the |
| | milk fermented at 37° C. |

Despite the fact that different pH values of the fermented milks make the comparative tasting a little hard, milk fermented at 37° C. for 8 hours was generally more appreciated than milk fermented at 42° C. for 6 hours. Moreover, a fermentation occurring at 37° C. is expected to favor lactobacillus growth over streptococcus growth, which is highly desirable in a probiotic product. Products having a final pH of more than 4.3 also present a stronger after-taste than those with lower pH values.

### Example 18

### Production of Fermented Milk with Adjusted Inoculation Values, and Evaluation of Bacterial Survival Rate in a Mango-Flavored Fermented Milk

One goal of this test was to evaluate the amount of lyophilized powder to be added to a milk containing 14% solids to obtain, following fermentation, a minimum of 1×10⁶ bacteria per gram of final product after fermentation and during storage for each of the seven strains added, and a final pH of about 4.00 to 4.40. Another goal was to evaluate the bacterial survival after storage at 7-8° C. of a final mango-flavored product.

Three samples, named A, B and C, of 800 g of reconstituted milk containing 14 total solids was prepared by adding 112 g of powdered skim milk to 688 g of running water and mixing for 10 minutes. Milk was pasteurized at 90° C. for 30 minutes in a bath before temperature was adjusted to 37° C. Inoculation of the 3 milk samples was performed according to table 14. Inoculation values were calculated from counting of lyophilized powders on MRS-sorbitol, except for bifidobacteria which were calculated with both MRS-sorbitol and NaLa-agar.

TABLE 14
Inoculation values of bacteria

| | Milk A | Milk B | Milk C |
|---|---|---|---|
| | (CFU/g) | (CFU/g) | (CFU/g) |
| S. thermophilus R-0083 | 1.00^{E}07 | 1.08^{E}06 | 1.03^{E}07 |
| L. bulgaricus 601383 | 9.00^{E}04 | 9.45^{E}04 | 9.00^{E}04 |
| L. acidophilus R-0052 | 1.07^{E}07 | 1.02^{E}07 | 1.0^{E}07 |
| L. casei 601379 | 1.00^{E}07 | 1.03^{E}07 | 9.99^{E}06 |
| B. longum 601377 | 1.76^{E}07 | 1.76^{E}07 | 8.9^{E}07 |
| B. lactis 601377 | 2.02^{E}07 | 2.0^{E}07 | 1.06^{E}08 |
| P. freudenreichii P63 | 9.18^{E}05 | 9.16^{E}05 | 8.97^{E}05 |

Fermentation was performed at 37° C. until a pH value of 4.2-4.3 was reached. Fermented milks A, B and C were then stored at 4° C. until the next day.

The milks were next flavored with mango flavor in a ration of 240 g of fermented milk for 148 g of fruit mix. Fruit mix was prepared by adding 192 g of mango puree (Delifruit™) to 96 g of apple concentrate (70° Brix, Jonhson Concentrate, lot AP625) in 156 g of water; pasteurization at 90° C. for 5 minutes; and cooling in an ice bath until a temperature of 10° C. was reached.

Differential counting of bacteria was performed after fermentation (and overnight cooling) of 5 samples of 30-40 ml from all three milks, and following mango flavoring of milk C. Counting conditions were as follow:
S thermophilus was counted on ST-agar at 37° C., aerobic;
L. bulgaricus, L. casei and L, acidophilus were counted on MRS (pH=4.58) at 37° C. for 72 h, anaerobic;
L. casei was counted on MRS-vancomycin-agar (1 mg/L) at 37° C. for 72 h, anaerobic; and,

B. bifidum and B. lactic were counted on MRS-NNLP with 0.05% cystein at 37° C. for 72 h, anaerobic.

Results are presented on tables 15 and 16. Fermentation time was about 6 hours for reaching desired pH values. All three milks showed little differences despite the fact that S. thermophilus concentration was reduced of about 10 times in milk B. Bacteria from milk C appeared to have produced as much acid than bacteria from milks A and B. Milk C had similar inoculation values to milk A, except that bifidobacteria were in higher proportions in milk C.

TABLE 15
pH evolution in unflavored and flavored fermented milks.

| | Fermentation | pH | pH | pH |
|---|---|---|---|---|
| Milk | time | before cooling | after cooling | after flavoring |
| A | 6 h 35 | 4.35 | 4.45 | 4.28 |
| B | 6 h 30 | 4.34 | 4.39 | 4.23 |
| C | 6 h 10 | 4.40 | 4.50 | 4.31 |

Results presented in table 16 showed that S. thermophilus and Lb. casei were present in expected amounts in the fermented milk at T=0. However, amounts of bifidobacteria were about 10 times less than expected, while Lb. bulgaricus and Lb. acidophilus, the counted amounts was very much under the expected amounts.

TABLE 16
Differential counting of bacteria at T = 0 for milk A.

| | Lyophilyzed | Quantity | Theoric | Counted |
|---|---|---|---|---|
| | powder | added to | CFU/g in | CFU/g in |
| | (CFU/g) | milk A (g) | milk A | milk A |
| S. thermophilus | 1.00 × 10¹¹ | 0.0802 | 1.00 × 10⁷ | 3.98 × 10⁷ |
| Lb. Bulgaricus | 1.20 × 10⁹ | 0.0600 | 9.00 × 10⁴ | <1.00 × 10⁴ |
| Lb. Acidophilus | 1.60 × 10¹¹ | 0.0537 | 1.07 × 10⁷ | <1.00 × 10⁴ |
| Lb. Casei | 1.00 × 10¹¹ | 0.0802 | 1.00 × 10⁷ | 1.00 × 10⁷ |
| B. longum | 1.10 × 10¹¹ | 0.1280 | 1.76 × 10⁷ | * |
| B. bifidum | 7.10 × 10¹⁰ | 0.2280 | 2.02 × 10⁷ | * |
| P. freudenreichii | 9.00 × 10⁹ | 0.0816 | 9.18 × 10⁵ | not counted |

* Differential counting method used did not allowed for distinct counting of those two bacteria. Combined count of B. longum and B. bifidum was of 5.01 × 10⁶.

Results presented in table 17 shows that milk C had the highest counts of bacteria after 24 hours. The minimum counts of 1.00×10⁷ ensure that even in the case where 1 log of bacteria was lost during subsequent storage, a minimum of 1 million viable bacteria from each species would still be present in the product.

TABLE 17
Differential counting of bacteria after completion of fermentation
of fermented milk and mango-flavored fermented milk.

| | | | Lb. acidophilus + | | |
|---|---|---|---|---|---|
| | | B. longum + | Lb. bulgaricus + | Lb. acidophilus + | |
| | S. thermophilus | B. lactis | Lb. casei | Lb. bulgaricus | Lb. casei |
| Sample | (CFU/g) | (CFU/g) | (CFU/g | (CFU/g | (CFU/g) |
| Milk A, T = 0 | 3.98 × 10⁷ | 5.01 × 10⁶ | 1.00 × 10⁴ | 9.99 × 10⁶ | 1.00 × 10⁷ |
| Milk A, T = 24 h | 1.99 × 10⁹ | 1.00 × 10⁵ | 1.00 × 10⁸ | 6.02 × 10⁷ | 3.98 × 10⁷ |
| Milk B, T = 24 h | 1.99 × 10⁹ | 5.01 × 10⁷ | 6.31 × 10⁷ | 4.32 × 10⁷ | 1.99 × 10⁷ |
| Milk C, T = 24 h | 1.00 × 10⁹ | 1.00 × 10⁸ | 2.51 × 10⁸ | 1.88 × 10⁸ | 6.31 × 10⁷ |
| Milk C | 1.00 × 10⁹ | 1.00 × 10⁸ | 1.00 × 10⁷ | 4.99 × 10⁶ | 5.01 × 10⁶ |
| (mango), T = 24 h | | | | | |

However, it seems that there is a loss in lactobacilli content following the addition of the mango-flavored mix. When considering that the mix contains 240 g of milk for 388 g of mix, with an average of 1.88×10⁸ CFU/g lactobacilli in milk, we should theoretically obtain about 1.16×10⁸ CFU/g in mango-flavored milk. Still, we counted only 4.99×10⁹ CFU/g of lactobacilli in mango-flavored milk. The loss is therefore more important than the one caused by the dilution with the addition of the fruit mix to the fermented milk. Since L. bulgaricus and L. acidophilus can not be differentiated when counting, we do not know which one is more effected by the decrease of 1 log in lactobacilli content.

Table 18 shows a great growth for S. thermophilus and combined growth for Lb. bulgaricus and Lb. acidophilus. However, due to the counting technique, it is unknown if both bacteria grew well or if one took over the other. Bifidobacteria and Lb. casei did not grow.

TABLE 18
Bacterial growth in milk C

| | Quantity added | Theoric CFU/g | Counted CFU/g |
|---|---|---|---|
| | to milk A (g) | in milk A | in milk A |
| S. thermophilus | 0.083 | 1.03 × 10⁷ | 1.00 × 10⁹ |
| Lb. bulgaricus | 0.060 | 9.00 × 10⁴ | * |
| Lb. acidophilus | 0.050 | 1.00 × 10⁷ | * |
| Lb. casei | 0.080 | 9.99 × 10⁶ | 1.00 × 10⁷ |
| B. longum | 0.647 | 8.90 × 10⁷ | ** |
| B. bifidum | 1.190 | 1.06 × 10⁸ | ** |
| P. freudenreichii | 0.080 | 8.97 × 10⁵ | not counted |

* Differential counting method used did not allowed for distinct counting of those two bacteria. Combined count of Lb. bulgaricus and Lb. acidophilus was of 1.88 × 10⁸.
** Differential counting method used did not allowed for distinct counting of those two bacteria. Combined count of B. longum and B. bifidum was of 1.00 × 10⁸.

The fermented milks have been tested for the presence of total coliforms. Contamination was too high after fermentation to allow for microbial analysis over time. Contamination has been identified as originating from the lyophilized powders. Bacterial survival can be affected by the presence of contaminants. Testing was nonetheless performed on fermented milks after 2 months storage at 4° C., with a VRBA analysis of total coliforms. Results from this test showed that no coliforms were present in the fermented milks after 2 months storage at 4° C.

### Example 19

### In Vitro Evaluation of the Survival Capacity of Probiotic Bacterial Strains from the BIACTIVE VIVACE™ Vanilla Product in Stomach and Small Intestine

BIACTIVE VIVACE™ vanilla product is a fermented milk comprising Streptococcus thermophilus, Lactobacillus acidophilus, Lactobacillus paracasei, Lactobacillus plantarum, Bifidobacterium lactis, Bifidobacterium longum and Propionibacterium freudenreichii as bacterial strains. Testing was performed using the TIM-1 model. For this test, the product was kept for 7 days at 4° C. to conservation time by a user prior to consumption. Duplicates originated from different fermentation batch. Selective media were used for the selective counting of bacterial strains present in the product during digestion.

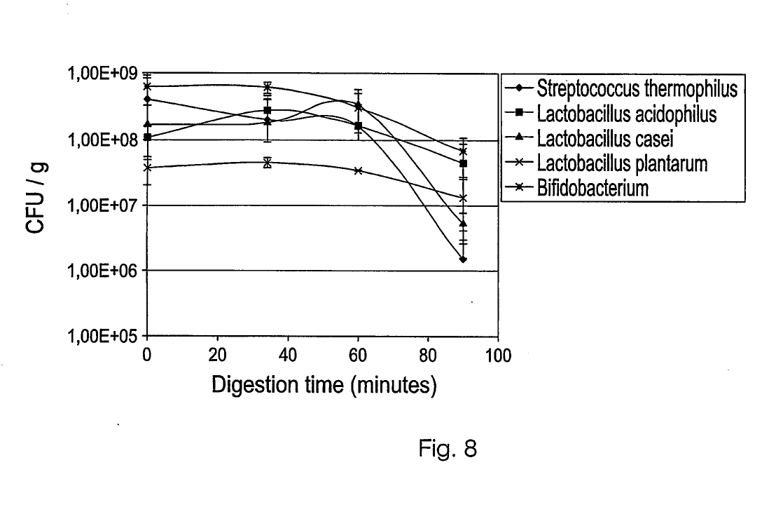

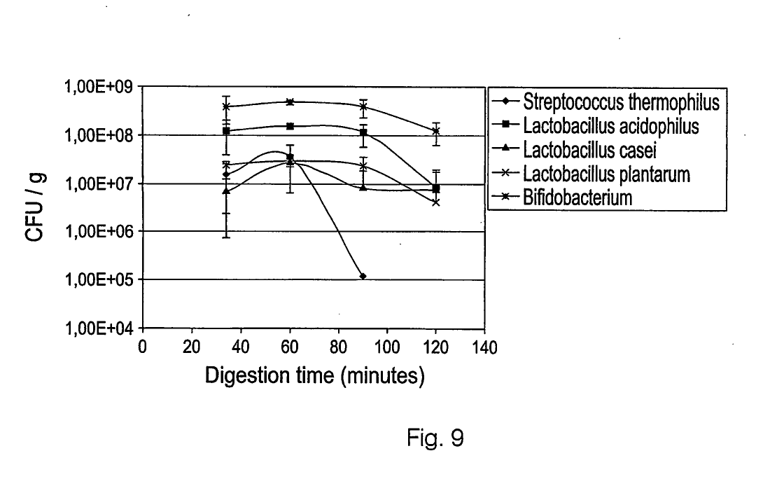

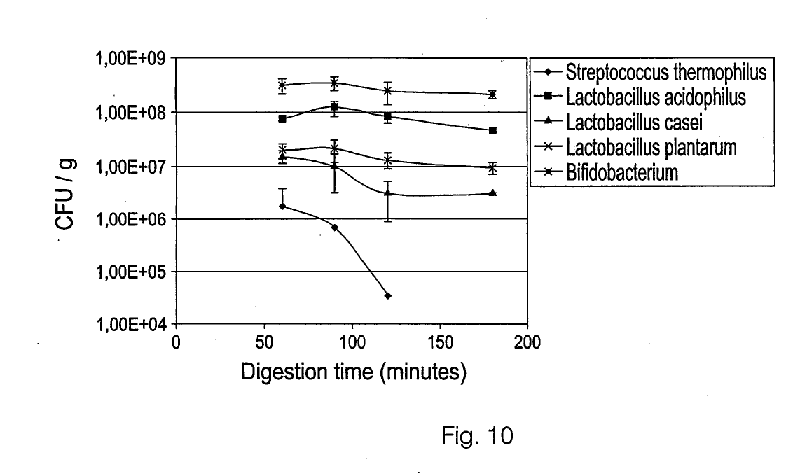

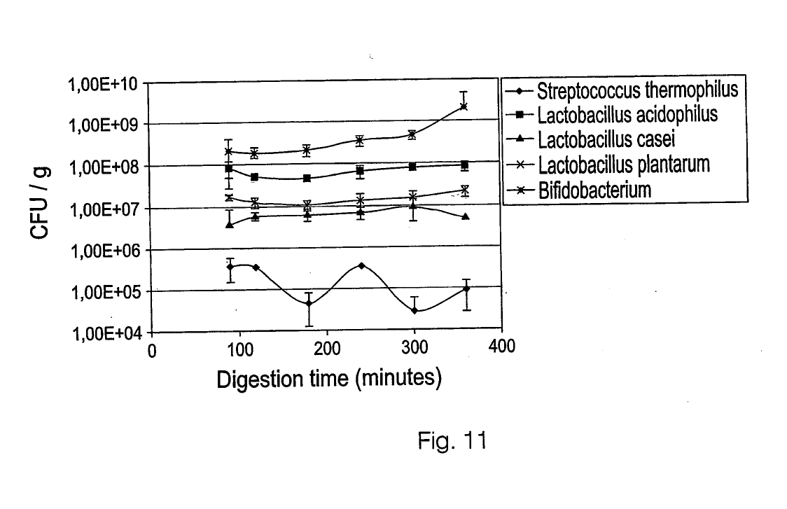

Results are shown in FIGS. 8 to 11. Most of the strains show an excellent survival rate over time in the different sections of TIM-1. The most important decrease occurs in the stomach after 90 minutes, with about 30% of the initial count still present. Bifidobacteria are the most resistant to the transit between the stomach to the small intestine. In the ileum, the mean survival rate for all the strains is of 81.5%, which correspond to a decrease of about 0.5 log CFU/g. Lb. acidophilus and Lb. plantarum also show a very good survival rate, corresponding to about 0.5 log CFU/g. FIGS. 8 to 10 show a good survival rate for Lb. casei paracasei, but FIG. 11 shows that the survival for this strain in the ileum is of between 6.5×10⁶ and 1.0×10⁷ CFU/g. Still, this number is sufficient for the strain to have beneficial effects. S. thermophilus had the lowest survival rate during GI transit, decreasing of about 4 log CFU/g. Counting for this strain had to be estimated in the ileum since the bacterial number was close to the detection threshold of the counting method.

Generally speaking, the survival capacity of the probiotic strains contained in the BIACTIVE VIVACE™ vanilla product is very good. Bifidobacteria strains BB12 and BB46, as well as Lb. acidophilus LA-5 and Lb. plantarum 601387 are particularly resistant to the stresses of GI transit. Lb. casei paracasei LC-01 presented a good survival rate, while S. thermophilus St-B01 presented the lowest survival rate in the stomach and small intestine. Inoculation values for S. thermophilus can not be increased without risking the viability and growth of other bacterial strains during fermentation.

While the invention has been described in connection with specific aspects thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A probiotic composition comprising a mixture of a Propionibacterium and at least one other probiotic bacteria species.

2. The probiotic composition of claim 1, wherein the other probiotic bacteria species is selected from the group comprising Lactobacillus, Bifidobacterium, and Streptococcus.

3. The probiotic composition of claim 1, wherein said probiotic composition comprises a mixture of living bacteria comprising a Propionibacterium, a Lactobacterium, a Bifidobacterium, and a Streptococcus.

4. The probiotic composition of claim 3, wherein said Lactobacterium is selected from the group comprising Lactobacillus acidophilus, Lactobacillus casei, Lactobacillus plantarum, Lactobacillus paracasei, Lactobacillus rhamnosus, Lactobacillus bulgaricus, Lactobacillus reuteri, Lactobacillus salivarius, and Lactobacillus crispatus.

5. The probiotic composition of claim 3, wherein said Bifidobacterium is selected from the group comprising Bifidobacterium longum, Bifidobacterium lactis, Bifidobacterium bifidum, Bifidobacterium infantis, and Bifidobacterium breve.

6. The probiotic composition of claim 3, said Streptococcus being Streptococcus thermophilus.

7. The probiotic composition of claim 1, said Propionibacterium being Propionibacterium freudenreichii.

8. The probiotic composition of claim 1 further comprising a prebiotic.

9. The probiotic composition of claim 8, wherein said prebiotic is at least one of inuline and oligofructose.

10. The probiotic composition of claims 1, wherein said probiotic composition comprises from between 10⁸ to 10¹² probiotic bacteria.

11. The probiotic composition of claim 1, comprising 10 to 30% of Lactobacillus acidophilus, 5 to 30% of Lactobacillus paracasei, 5 to 30% of Bifidobacterium lactis, 5 to 30% of Bifidobacterium longum, 5 to 20% of Bifidobacterium bifidum, 5 to 20% of Bifidobacterium breve, 5 to 30% of Bifidobacterium infantis, 5 to 30% of Propionibacterium freudenreichii, and 2 to 20% of Streptococcus thermophilus.

12. The probiotic composition of claim 11, wherein said Lb. acidophilus is present at a concentration of about 15%, Lb. casei paracasei is present at a concentration of about 15%, B. lactis is present at a concentration of about 15%, B. longum is present at a concentration of about 10%, B. bifidum is present at a concentration of about 10%, B. breve is present at a concentration of about 10%, B. infantis is present at a concentration of about 10%, P. freudenreichii is present at a concentration of about 10% and S. thermophilus is present at a concentration of about 5%.

13. The probiotic composition of claim 1, comprising 10 to 30% of Lactobacillus rhamnosus, 10 to 30% of Lactobacillus acidophilus, 10 to 30% of Lactobacillus paracasei, 10 to 30% of Lactobacillus plantarum, 5 to 20% of Bifidobacterium bifidum, 5 to 20% of Bifidobacterium breve, 5 to 15% of Streptococcus thermophilus, and 5 to 20% of Propionibacterium freudenreichii.

14. The probiotic composition of claim 13, wherein said Lactobacillus rhamnosus is present at a concentration of about 20%, Lactobacillus acidophilus is present at a concentration of about 20%, Lactobacillus paracasei is present at a concentration of about 15%, Lactobacillus plantarum is present at a concentration of about 15%, Bifidobacterium bifidum is present at a concentration of about 10%, Bifidobacterium breve is present at a concentration of about 10%, Streptococcus thermophilus is present at a concentration of about 5% and Propionibacterium freudenreichii is present at a concentration of about 5%.

15. The probiotic composition of claim 1, comprising 10 to 30% of Lactobacillus rhamnosus, 10 to 30% of Lactobacillus plantarum, 10 to 30% of Lactobacillus acidophilus, 5 to 20% of Lactobacillus paracasei, 5 to 20% of Propionibacterium freudenreichii, 5 to 20% of Bifidobacterium lactis, 5 to 10% of Bifidobacterium bifidum, 5 to 10% of Bifidobacterium breve, 5 to 15% of Bifidobacterium infantis, 3 to 10% of Bifidobacterium longum, 2 to 10% of Lactobacillus bulgaricus, and 2 to 10% of Streptococcus thermophilus.

16. The probiotic composition of claim 15, wherein said Lactobacillus rhamnosus is present at a concentration of about 18%, Lactobacillus plantarum is present at a concentration of about 18%, Lactobacillus acidophilus is present at a concentration of about 10%, Lactobacillus paracasei is present at a concentration of about 10%, Propionibacterium freudenreichii is present at a concentration of about 10%, Bifidobacterium lactis is present at a concentration of about 10%, Bifidobacterium bifidum is present at a concentration of about 5%, Bifidobacterium breve is present at a concentration of about 5%, Bifidobacterium infantis is present at a concentration of about 5%, Bifidobacterium longum is present at a concentration of about 4%, Lactobacillus bulgaricus is present at a concentration of about 2.5% and Streptococcus thermophilus is present at a concentration of about 2.5%.

17. A method for normalizing the gastrointestinal flora in a subject in need, said method comprising administering the probiotic composition of claim 1 to the subject.

18. A method for preventing and treating a gastrointestinal disorder in a subject in need thereof, said method comprising administering the probiotic composition of claim 1 to the subject.

19. Use of the probiotic composition of claim 1 in the manufacture of a preparation for the normalization of a gastrointestinal flora in a subject.

20. Use of the probiotic composition of claim 1 for the normalization of a gastro-intestinal flora in a subject.

21. Use of the probiotic composition of claim 1 for the manufacture of a preparation for the prevention and treatment of a gastrointestinal disorder in a subject.

22. Use of the probiotic composition of claim 1 for the prevention and treatment of a gastrointestinal disorder in a subject.

23. Use of the probiotic composition of claim 1 for the manufacture of a food supplement.

24. The probiotic composition of claim 1, said composition being formulated as a solid.

25. The probiotic composition of claim 24, wherein said solid is a lyophilized powder packed in a capsule.

26. A method for producing a probiotic composition, said method comprising mixing a Propionibacterium with at least one other probiotic bacterial species, thereby obtaining said probiotic composition.

27. The method of claim 26, wherein the other probiotic bacteria species is selected from the group comprising Lactobacillus, Bifidobacterium, and Streptococcus.
